(19) United States
(12) Patent Application Publication
(10) Pub. No.: US 2024/0094202 A1
(43) Pub. Date: Mar. 21, 2024

(54) Detection, Treatment, and Monitoring of Microbiome-Dependent Gastrointestinal Discomfort

(71) Applicant: McPharma Biotech Inc., Carberry (CA)

(72) Inventors: Derek McLaren, Carberry (CA); Earl McLaren, Carberry (CA)

(21) Appl. No.: 18/031,475

(22) PCT Filed: Oct. 14, 2021
(86) PCT No.: PCT/CA2021/051443

§ 371 Date: Apr. 12, 2023

Related U.S. Application Data
(60) Provisional application No. 63/092,605, filed on Oct. 16, 2020.

(51) Int. Cl. G01N 33/569

(52) U.S. Cl. CPC G01N 33/56911; G01N 2800/065; G01N 2800/52

(57) ABSTRACT

Irritable Bowel Syndrome (IBS) is a multifactorial disorder characterized by pain, bloat, and changes in bowel movements, where different bacteria in the gut microbiome potentially influence some or all of the related symptoms. However, IBS is extremely heterogenous, suggesting a need to identify relationships between groups of bacteria in the gut microbiome and specific IBS-related symptoms. RS consumption significantly altered the microbiome while improving several measures of IBS. The use of RS by those needing to reduce the IBS-related symptoms, including those diagnosed with IBS, can be guided by simultaneously measuring changes in select genera in the microbiome.

### PRIOR APPLICATION INFORMATION

The instant application claims the benefit of U.S. Provisional Patent Application Ser. No. 63/092,605, filed Oct. 16, 2020 and entitled “Detection, treatment, and monitoring of microbiome-dependent gastrointestinal discomfort”, the entire contents of which are incorporated herein by reference for all purposes.

### BACKGROUND OF THE INVENTION

Irritable Bowel Syndrome (IBS) is a complex gastrointestinal disorder characterized by a constellation of overlapping, self-reported digestive symptoms, including diarrhea, constipation, bloating, abdominal pain, and/or gas. Diagnosis is challenging due to the absence of objective pathologies, such as lesions or elevated inflammatory markers. Indeed, the presence of colonic lesions and inflammation in individuals with IBS-like digestive symptoms would trigger a diagnosis of inflammatory bowel disease (IBD) rather than IBS. Consequently, people suffering from IBS-related symptoms typically undergo diagnostic tests and procedures to exclude conditions like IBD and celiac disease, and only obtain an IBS diagnosis when all other tests are negative.

Rome IV Diagnostic Questionnaires are currently the gold standard for diagnosing functional gastrointestinal disorders, including 62.7% sensitivity and 97.1% specificity for IBS (Palsson et al. 2016. Gastroenterology), highlighting the importance of subject-reported questionnaire-based data in making a diagnosis. IBS diagnosis relies on patients meeting a minimum frequency of symptoms, such that people with less frequent IBS-related symptoms go undiagnosed. Symptom heterogeneity in IBS has led to the stratification of patients into sub-categories: Constipation predominant (IBS-C), diarrhea predominant (IBS-D), or where symptoms are mixed or alternate between these extremes (IBS-M). The etiology of IBS in general and the sub-categories specifically, is unknown.

Recent advances in microbiological research methods, namely the ability to identify and characterize unculturable bacteria in the digestive tract, has spurred investigation into the relationship between the composition of the gut microbiome and IBS. The role of elevated levels of Proteobacteria, including family Enterobacteriaceae, was emphasized in a recent meta-analysis of IBS studies reporting gut microbiome findings (Pittayanon et al. 2019. Gastroenterology), but a unifying hypothesis linking IBS symptomology to a discrete microbiome pattern remains elusive.

It is not surprising that overlaying the complex heterogeneity of the gut microbiome with the heterogenous symptoms of IBS failed to yield generalizable conclusions. While presence/absence differences for certain bacteria may facilitate understanding of disease etiology in some conditions, the relationship between IBS symptoms and bacteria is likely dynamic, with patterns only emerging under treatment modalities where both the microbiome and symptom levels changes.

We hypothesized that different bacteria may be responsible for various IBS symptoms, and that measuring changes in both the abundance of bacteria and magnitude of symptoms in healthy people consuming a prebiotic would reveal important correlations. Our findings indicate that microbiome patterns can be linked to different IBS symptoms. As discussed below, these patters emerge following microbiome modulating treatment(s) that provide symptom relief. That is, described below is how the microbiome patterns contributing to or responsible for the IBS symptoms were determined following treatment of the symptoms. As discussed herein, these gut microbiome patterns can be considered as “fingerprints”, that is, specific characteristics within the gut microbiome profile indicative of potential treatment with microbiome modulating treatments.

### SUMMARY OF THE INVENTION

According to an aspect of the invention, there is provided a method for determining efficacy of a gut microbiome modulating treatment for bloating in an individual at risk of developing or who has developed or who has IBS, said method comprising:
detecting the levels of Haemophilus, Pasteurellaceae, Enterobacteriaceae, Gammaproteobacteria, Granulicatella, Lachnospira, Oribacterium, Fusobacteriaceae not belonging to the genera Cetobacterium, Fusobacterium, Propionigenium, Psychrilyobacter, or u114 (hereafter ‘select Fusobacteriaceae), Alistipes, Parabacteroides, Subdoligranulum, and/or Eggerthella in a first gut microbiome sample from the individual at a first time point;
administering to the individual a microbiome modulating treatment on a dosage regimen for a suitable period of time;
following the suitable period of time, obtaining a second gut microbiome sample from the individual;
detecting the levels of Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, and/or Eggerthella in the second sample; and
comparing the levels of a given bacterium/bacteria in the second gut microbiome sample to levels of those same bacterium/bacteria in the first gut microbiome sample,
wherein if the Haemophilus, Pasteurellaceae, Enterobacteriaceae, Gammaproteobacteria, Granulicatella, Lachnospira, Oribacterium, and/or select Fusobacteriaceae levels in the second sample are lower than levels of those same bacterium/bacteria in the first sample, and/or the Alistipes, Parabacteroides, Subdoligranulum, and/or Eggerthella levels in the second sample are higher than levels of those same bacterium/bacteria in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating treatment for bloating in an individual at risk of developing or who has developed or who has IBS, said method comprising:
detecting levels of Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, and/or Eggerthella in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting the levels of Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, and/or Eggerthella in the second sample;
determining a second measurement of the IBS-related parameter of the individual at the second time point;
comparing the levels of a given bacterium/bacteria in the second gut microbiome sample to levels of those same bacterium/bacteria in the first gut microbiome sample, and
comparing the first measurement of the IBS-related parameter and the second measurement of the IBS-related parameter,
wherein if the Haemophilus, Pasteurellaceae, Enterobacteriaceae, Gammaproteobacteria, Granulicatella, Lachnospira, Oribacterium, and/or select Fusobacteriaceae levels in the second sample are lower than levels of those same bacterium/bacteria in the first sample, and/or the Alistipes, Parabacteroides, Subdoligranulum, and/or Eggerthella levels in the second sample are higher than levels of those same bacterium/bacteria in the first sample and the second IBS-related parameter is improved compared to the first IBS-related parameter, continuing the dosage regimen for the individual.

According to another aspect of the invention, there is provided a method for determining efficacy of a microbiome modulating treatment of constipation in an individual at risk of developing or who has developed or who has IBS, said method comprising:
detecting Gammaproteobacteria, Granulicatella, and/or Alistipes levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Gammaproteobacteria, Granulicatella, and/or Alistipes levels in the second sample; and
comparing the levels of a given bacterium/bacteria in the second gut microbiome sample to levels of those same bacterium/bacteria in the first gut microbiome sample,
wherein if the Gammaproteobacteria and/or Granulicatella levels in the second sample are lower than the levels in the first sample and/or Alistipes levels in the second sample are higher than the levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating of constipation in an individual at risk of developing or who has developed or who has IBS, said method comprising:
detecting Gammaproteobacteria, Granulicatella, and/or Alistipes levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting Gammaproteobacteria, Granulicatella, and/or Alistipes levels in the second sample;
determining a second measurement of the IBS-related parameter of the individual at the second time point;
comparing the levels of a given bacterium/bacteria in the second gut microbiome sample to levels of those same bacterium/bacteria in the first gut microbiome sample, and
comparing the first measurement of the IBS-related parameter and the second measurement of the IBS-related parameter,
wherein if the Gammaproteobacteria and/or Granulicatella levels in the second sample are lower than the levels in the first sample and/or Alistipes levels in the second sample are higher than the levels in the first sample, and the second IBS-related parameter is improved compared to the first IBS-related parameter, continuing the dosage regimen for the individual.

According to another aspect of the invention, there is provided a method for determining efficacy of a microbiome modulating treatment of abdominal pain in an individual at risk of developing or who has developed or who has IBS, said method comprising:
detecting Proteobacteria belonging to classes Acidithiobacilia, Hydrogenophilalia, Oligoflexia, and/or other classes (hereafter ‘select Proteobacteria’) levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting select Proteobacteria levels in the second sample; and
comparing the levels of select Proteobacteria in the second gut microbiome sample to levels of select Proteobacteria in the first gut microbiome sample,
wherein if the select Proteobacteria levels in the second sample are higher than the select Proteobacteria levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating of abdominal pain in an individual at risk of developing or who has developed or who has IBS, said method comprising:
detecting select Proteobacteria levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting select Proteobacteria levels in the second sample;
determining a second measurement of the IBS-related parameter of the individual at the second time point;
comparing the levels of select Proteobacteria in the second gut microbiome sample to levels of select Proteobacteria in the first gut microbiome sample, and
comparing the first measurement of the IBS-related parameter and the second measurement of the IBS-related parameter,
wherein if the select Proteobacteria levels in the second sample are higher than the select Proteobacteria levels in the first sample, and the second IBS-related parameter is improved compared to the first IBS-related parameter, continuing the dosage regimen for the individual.

According to another aspect of the invention, there is provided a method for determining efficacy of a microbiome modulating treatment of diarrhea in an individual at risk of developing or who has developed or who has IBS, said method comprising:
detecting Ruminococcus levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Ruminococcus levels in the second sample; and
comparing the levels of Ruminococcus in the second gut microbiome sample to levels of Ruminococcus in the first gut microbiome sample,
wherein if the Ruminococcus levels in the second sample are higher than the Ruminococcus levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating of diarrhea in an individual at risk of developing or who has developed or who has IBS, said method comprising:
detecting Ruminococcus levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting Ruminococcus levels in the second sample;
determining a second measurement of the IBS-related parameter of the individual at the second time point;
comparing the levels of Ruminococcus in the second gut microbiome sample to levels of Ruminococcus in the first gut microbiome sample, and
comparing the first measurement of the IBS-related parameter and the second measurement of the IBS-related parameter,
wherein if the Ruminococcus levels in the second sample are higher than the Ruminococcus levels in the first sample, and the second IBS-related parameter is improved compared to the first IBS-related parameter, continuing the dosage regimen for the individual.

According to another aspect of the invention, there is provided a method for determining efficacy of a microbiome modulating treatment of belching in an individual at risk of developing or who has developed or who has IBS, said method comprising:
detecting levels of Bacteroidaceae bacteria not belonging to genera 5-7N15, Bacteroides, or BF311 (hereafter ‘select Bacteroidaceae’) in a first gut microbiome sample from the individual at a first time point;
administering to the individual a microbiome modulating treatment on a dosage regimen for a suitable period of time;
following the suitable period of time, obtaining a second gut microbiome sample from the individual;
detecting select Bacteroidaceae levels in the second sample; and
comparing the levels of select Bacteroidaceae in the second gut microbiome sample to levels of select Bacteroidaceae in the first gut microbiome sample,
wherein if the select Bacteroidaceae levels in the second sample are lower than the select Bacteroidaceae levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating of belching in an individual at risk of developing or who has developed or who has IBS, said method comprising:
detecting select Bacteroidaceae levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting select Bacteroidaceae levels in the second sample;
determining a second measurement of the IBS-related parameter of the individual at the second time point;
comparing the levels of select Bacteroidaceae in the second gut microbiome sample to levels of select Bacteroidaceae in the first gut microbiome sample, and
comparing the first measurement of the IBS-related parameter and the second measurement of the IBS-related parameter,
wherein if the select Bacteroidaceae levels in the second sample are lower than the select Bacteroidaceae levels in the first sample, and the second IBS-related parameter is improved compared to the first IBS-related parameter, continuing the dosage regimen for the individual.

According to another aspect of the invention, there is provided a method for determining efficacy of a microbiome modulating treatment of overall well-being in an individual at risk of developing or who has developed or who has IBS, said method comprising:
detecting Anaerostipes and/or Mogibacterium levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Anaerostipes and/or Mogibacterium levels in the second sample; and
comparing the levels of Anaerostipes and/or Mogibacterium in the second gut microbiome sample to levels of Anaerostipes and/or Mogibacterium in the first gut microbiome sample,
wherein if the Anaerostipes and/or Mogibacterium levels in the second sample are lower than the Anaerostipes and/or Mogibacterium levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating of overall well-being in an individual at risk of developing or who has developed or who has IBS, said method comprising:
detecting Anaerostipes and/or Mogibacterium levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting Anaerostipes and/or Mogibacterium levels in the second sample;
determining a second measurement of the IBS-related parameter of the individual at the second time point;
comparing the levels of Anaerostipes and/or Mogibacterium in the second gut microbiome sample to levels of Anaerostipes and/or Mogibacterium in the first gut microbiome sample, and
comparing the first measurement of the IBS-related parameter and the second measurement of the IBS-related parameter,
wherein if the Anaerostipes and/or Mogibacterium levels in the second sample are lower than the Anaerostipes and/or Mogibacterium levels in the first sample, and the second IBS-related parameter is improved compared to the first IBS-related parameter, continuing the dosage regimen for the individual.

In some embodiments, the gut microbiome sample is, for example but by no means limited to, a stool or fecal sample or colonic contents, whether sampled in situ or via intervention.

In some embodiments, the microbiome modulating treatment is a microbiome therapy, that is, a treatment that is known to or expected to alter the microbiome of the individual. Examples of microbiome therapies are discussed herein and other examples will be readily apparent to one of skill in the art.

According to another aspect of the invention, there is provided a method for detecting the signature or ‘fingerprint’ of an altered gut microbiome (also known as dysbiosis) that is correlated with IBS-related symptoms in an individual comprising the monitoring of Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, Eggerthella, select Proteobacteria, Ruminococcus, select Bacteroidaceae, Anaerostipes and/or Mogibacterium levels and predicting the efficacy of microbiome therapies if Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, Eggerthella, select Proteobacteria, Ruminococcus, select Bacteroidaceae, Anaerostipes and/or Mogibacterium are present.

As will be appreciated by one of skill in the art, levels of bacteria in the gut microbiome may be detected in a sample by a variety of means, which will be readily apparent to one of skill in the art. Illustrative examples are provided below.

In some embodiments of the invention, Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, Eggerthella, select Proteobacteria, Ruminococcus, select Bacteroidaceae, Anaerostipes and/or Mogibacterium is/are detected by directed 16S V4 ribosomal subunit amplification (for example, Real-Time Polymerase Chain Reaction; RT-PCR or Quantitative PCR; qPCR) of each bacterial group using the abundance of Bacteroides or other common commensal unrelated to IBS-related symptoms as the reference value. As will be apparent to one of skill in the art, Bacteroides is both common (found in most gut microbiomes) and abundant (making up a large proportion of each microbiome), and accordingly is suitable to be used as an internal control. However, other suitable candidates for use as an internal control will be readily apparent to one of skill in the art.

In another embodiment of the invention, Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, Eggerthella, select Proteobacteria, Ruminococcus, select Bacteroidaceae, Anaerostipes and/or Mogibacterium is detected by whole microbiome sequencing using the 16S V4 ribosomal subunit and/or other relevant regions.

In another embodiment of the invention, Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, Eggerthella, select Proteobacteria, Ruminococcus, select Bacteroidaceae, Anaerostipes and/or Mogibacterium is detected by shotgun metagenome sequencing, or another suitable unbiased genomic-based approach, or any method that reports proportional representation of Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, Eggerthella, select Proteobacteria, Ruminococcus, select Bacteroidaceae, Anaerostipes and/or Mogibacterium in the microbiome.

In some embodiments of the invention, the individual has abdominal pain, including bloating or abdominal distension, approximately one day per week, related to increases or decreases in pain with defecation, pain associated with changes in stool frequency, and/or pain associated with changes in stool shape.

In another embodiment of the invention, the individual is at risk of developing IBS due to family history, lifestyle factors, or due to co-morbidities such as anxiety or depression.

In another embodiment of the invention, the individual has bloating with or without pain; suffers from diarrhea, constipation, belching, gas (ie. flatus); or suffers impairments to overall well-being, including mental wellness, anxiety, depression, fatigue, or sleeplessness.

In another embodiment of the invention, the individual has been diagnosed with or is suspected of having IBS.

In some embodiments of the invention, the microbiome therapy is a prebiotic, administered daily or as needed, for as long as the IBS-related markers continue to show improvement compared to baseline levels.

As discussed herein, the prebiotic microbiome therapeutic may be digestion resistant starch from potatoes or resistant potato starch, delivered daily or as needed, for as long as the IBS-related markers continue to show improvement compared to baseline levels.

As discussed herein, the effective amount may be for example 2 to 40 g, 2 to 30 g, 2 to 20 g, 5 to 40 g, 5 to 30 g, 5 g to 20 g, or 10 to 20 g of resistant potato starch.

In another embodiment of the invention, the microbiome therapy is a probiotic, administered daily or as needed, for as long as the IBS-related markers continue to show improvement compared to baseline levels.

In another embodiment of the invention, the microbiome therapy is an antibiotic, administered daily or as needed, for as long as the IBS-related markers continue to show improvement compared to baseline levels.

In another embodiment of the invention, the microbiome therapy is a combination of prebiotics, and/or probiotics, and/or antibiotics, and/or bacteriophages, administered daily or as needed, for as long as the IBS-related markers continue to show improvement compared to baseline levels.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

We sought to understand the relationship between IBS-related symptoms and the gut microbiome while circumventing the potential confounding effects of dysbiosis in IBS patients. To this end, we investigated the effects of placebo, 3.5 g, or 7 g per day of resistant potato starch (RPS) on self-reported IBS-related symptoms, including diarrhea, constipation, bloating, abdominal pain, gas, belching, and overall well-being, in healthy adults (ie. No IBS diagnosis). 6,006 correlations between changes in IBS-related symptoms and changes in the gut microbiome in people consuming placebo, 3.5 g or 7 g RPS daily after 1 or 4 weeks were calculated. At a minimum, correlations between a symptom and genus of bacteria were considered significant if they were directionally shared between time points at 7 g/day and/or between doses with p<0.05 for the first and p<0.1 for the second correlation (probability of Type 1 error: 1:200).

The relationship between shared correlations provided insight into how RPS affected the symptoms: Correlations shared between placebo and two or more treatments (n=4) were assumed to be independent of treatment unless the treatment p value was lower than that of the placebo, correlations shared between both placebo timepoints (n=4) were considered to be due to the placebo, correlations shared between both 7 g timepoints (n=12) were considered to be dose-dependent, correlations shared between 3.5 g and 7 g doses at 4 weeks (n=1) were considered to be duration-dependent, and correlations shared between 3 of 4 or all four treatments (n=4) were considered to be due to RPS treatment independent of time or dose.

Shared correlations were deemed spurious if they met the following conditions: Correlations with different directionality (n=14; unclear how opposing effects could be significantly meaningful), correlations that were shared at one week but not at four weeks (n=3; unclear how significant correlations could be lost over time), correlations shared at weeks one and four at 3.5 g/day (n=5; unclear how a low dose creates an effect but a high dose cannot), correlations shared between placebo and the 3.5 g/day dose (n=9; unclear if effect is general or due to shared consumption of placebo material, as 3.5 g dose contains 3.5 g RPS and 3.5 g placebo), or correlations shared between placebo and a 7 g/day dose (n=1; unclear how correlations shared between only two unrelated groups constitute a relationship independent of treatment).

Microbiome-symptom correlations for each group of bacteria were categorized by symptom. Thirteen bacterial groups were correlated with changes in bloating (Table 1). RPS consumption led to reductions in bloating associated with reductions in Gammaproteobacteria, including Enterobacteriaceae, Pasteurellaceae, and Haemophilus, increases in Bacteroidales genera Alistipes and Parabacteroides, and decreases in Lachnospiraeae genera Lachnospira and Oribacterium. Increases in Subdoligranulum and Eggerthella were associated with reductions in bloating in those consuming RPS, as were decreases in Granulicatella and Fusobacteriaceae. Decreases in RFN20 were correlated with increases in bloating in the placebo group.

RPS reduced constipation by decreasing Gammaproteobacteria and Granulicatella, while increasing Alistipes (Table 2). Notably, the constipation-microbiome correlations induced by RPS are consistent with those shared with bloating (Table 1), which further supports these observations given that bloating is often reported alongside constipation and that this gut microbiome ‘fingerprint’ helps distinguish constipation and bloating from other IBS symptoms. However, RPS-dependent increases in unclassified Proteobacteria were correlated with reductions in abdominal pain as were placebo-dependent increases in Oxalobacteraceae (Table 3), supporting the notion that not all Proteobacteria are necessarily harmful.

The sole gas-microbiome correlation was independent of interventions and suggested that increases in cyanobacteria were correlated with increases in gas (Table 4). The sole diarrhea-microbiome correlation was also independent of interventions, though the correlation between increases in Ruminococcus and decreases in diarrhea was more pronounced in RPS-consuming groups (Table 5).

In addition to symptoms typically reported by people suffering from IBS, we also measured changes in belching and overall well-being. Decreasing levels of Bacteroidaceae were correlated with RPS-dependent reductions in belching while placebo-dependent decreases in Dehalobacterium were correlated with increases in belching (Table 6). RPS-dependent decreases in levels of Anaerostipes and Mogibacterium, both members of order Eubacteriales, were correlated with increases in well-being, while placebo-dependent decreases in Victivallis were correlated with decreases in well-being (Table 7).

The orphan bacteria assigned to ‘other’ taxonomic units at the Family or higher level presented a problem because it was unclear whether general changes in this higher taxonomic group had predictive value or whether the predictive value lay only with a subset of the taxonomic group that had incompletely been described and therefore lacked a more specific name. To this end, we collapsed groups containing the ‘other’ qualification by collecting all genera identified in our study that belonged to the category, and summed the changes in abundance. This created data sets for changes in the abundance of the following taxonomic groupings: Proteobacteria (Phylum), Gammaproteobacteria (Class), Enterobacteriaceae (Family), Pasteurellaceae (Familly), Fusobacteriaceae (Family), and Bacteroidaceae (Family). We then generated Pearson correlation coefficients between changes in bacteria and changes in IBS-related symptoms, and tested their significance to see if the correlations identified at finer resolutions held at a coarser level (Table 8).

Correlations between IBS-related symptoms and Gammaproteobacteria, Enterobacteriaceae, and Pasteurellaceae retained significance, supporting the conclusion that monitoring changes in Classes (Gammaproteobacteria) or Families (Enterobacteriaceae and Pasteurellaceae) of bacteria is sufficient to predict improvements in IBS-related symptoms. Significant correlations were detected between Proteobacteria (Phylum) and abdominal pain, and between Fusobacteriaceae (Family) and bloating, but these associations were lost at the later time point. Fortunately, the Greengenes database (version 13-8-99) contains a list of genera belonging to Family Fusobacteria that allow us to further refine ‘other Fusobacteria’ to specifically state ‘Fusobacteria not belonging to the genera Cetobacterium, Fusobacterium, Propionigenium, Psychrilyobacter, or u114, hereafter known as ‘select Fusobacteriaceae’.

It is further noted that, as used herein in regards specific families and phyla of bacteria, the expression “select” or “other” can also be understood to mean “unidentified” or “unidentifiable”. Specifically, as discussed herein, in instances wherein there is a correlation between a specific symptom and either a “select” or “other” group of bacteria, it should be understood that this “select” or “other” group represents “unidentified” or “unidentifiable” bacteria strains, as discussed herein.

There were no significant correlations between Bacteroidaceae and belching, suggesting that ‘other Bacteroidaceae’ be revised to Bacteroidaceae not belonging to genera 5-7N15, Bacteroides, or BF311 , hereafter known as ‘select Bacteroidaceae’.

We attempted to revise the list of bacteria in the Proteobacteria (Phylum) group to see if there was a way to understand which bacteria in this group are responsible for the relationship. We noted that there was an inverse relationship between increases in Proteobacteria and decreases in abdominal pain, which was opposite to correlations reported between reductions in IBS-related symptoms in reductions in Proteobacteria belonging to Gammaproteobacteria, Enterobacteriaceae, and Pasteurellaceae. We therefore asked whether the correlation between increasing Proteobacteria and decreasing abdominal pain retained significance if the confounding effects of Gammaproteobacteria, including Enterobacteriaceae and Pasteurellaceae, were removed. To this end, we subtracted the changes in Enterobacteriaceae, Pasteurellaceae, and other Gammaproteobacteria from changes in all other Proteobacteria. This refinement failed to generate improvements over the correlation between changes Proteobacteria (Phylum) and changes in abdominal pain, suggesting that the bacteria identified here as ‘other Proteobacteria’ can be defined as those bacteria belonging to classes Acidithiobacilia, Hydrogenophilalia, Oligoflexia, and/or other classes, which are known to belong to phylum Proteobacteria (List of Prokaryotic names with Standing Nomenclature; https://lpsn.dsmz.de/phylum/proteobacteria—accessed Oct. 13, 2020) but are absent from the Greengenes database version 13-8-99, which are responsible for reducing abdominal pain in response to RS consumption.

As discussed below, RPS consumption significantly altered the microbiome while improving several measures of protein fermentation in the gut. The use of RPS by those needing to reduce the effects of protein fermentation, for example patients with chronic kidney disease, can be guided by simultaneously measuring changes in select genera in the microbiome

MSP Starch Products Inc. manufactures MSPrebiotic® Resistant Potato Starch, an unmodified type 2 resistant starch (RS2) that is a Solanum tuberosum preparation of food grade quality for animal and human food application. Resistant potato starch is also referred to as digestion or digestive resistant starch, or simply resistant starch (RS). While MSPrebiotic®, which contains 70% fiber, is used in the trials and experiments discussed herein, it is important to note that as discussed herein, another suitable resistant potato starch or potato resistant starch, that is, another unmodified RS type 2 potato starch, comprising at least 60% resistant starch or at least 65% resistant starch or at least 70% resistant starch or at least 75% resistant starch or at least 80% resistant starch of total extract or total potato extract may be used. That is, the extract itself may comprise at least 60% resistant starch, at least 65% resistant starch, at least 70% resistant starch, at least 75% resistant starch or at least 80% resistant starch on a weight to weight basis.

According to an aspect of the invention, there is provided a method for determining efficacy of a gut microbiome modulating treatment for bloating in an individual at risk of developing or who has developed or who has IBS, said method comprising:
detecting baseline levels of at least one gut microbiome bacteria selected from the group consisting of Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, and Eggerthella in a baseline gut microbiome sample from the individual at a first time point;
administering to the individual a microbiome modulating treatment on a dosage regimen for a suitable period of time;
following the suitable period of time, obtaining a treatment gut microbiome sample from the individual;
detecting treatment levels of the at least one gut microbiome bacteria in the treatment sample; and
comparing the levels of the at least one gut microbiome bacteria in the treatment gut microbiome sample to the baseline levels of said at least one gut microbiome bacteria in the baseline gut microbiome sample,
wherein if the Haemophilus, Pasteurellaceae, Enterobacteriaceae, Gammaproteobacteria, Granulicatella, Lachnospira, Oribacterium, and/or select Fusobacteriaceae levels in the treatment sample are lower than the baseline levels, and/or the Alistipes, Parabacteroides, Subdoligranulum, and/or Eggerthella levels in the treatment sample are higher than the baseline levels , continuing the dosage regimen for the individual.

As will be appreciated by one of skill in the art, whichever at least one gut microbiome bacteria are selected for detection for the baseline levels, those same bacteria are selected for detection in the treatment levels. As such, these may be referred to herein as “baseline levels of the at least one gut microbiome bacteria” and “treatment levels of the corresponding or respective at least one gut microbiome bacteria” if necessary to indicate that the levels of the same bacteria are being compared, although it is believed that this will be clear to one of skill in the art.

In some embodiments, at the first time point and the second time point, at least one IBS-related parameter of the individual is measured and these two measurements are also compared.

According to another aspect of the invention, there is provided a method for determining efficacy of a microbiome modulating treatment of constipation in an individual at risk of developing or who has developed or who has IBS, said method comprising:
detecting Gammaproteobacteria, Granulicatella, and/or Alistipes baseline levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Gammaproteobacteria, Granulicatella, and/or Alistipes treatment levels in the second sample; and
comparing the baseline levels to the treatment levels,
wherein if the Gammaproteobacteria and/or Granulicatella treatment levels are lower than the baseline levels and/or Alistipes treatment levels are higher than the baseline levels, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating treatment of abdominal pain in an individual at risk of developing or who has developed or who has IBS, said method comprising:
detecting select Proteobacteria (defined as belonging to classes Acidithiobacilia, Hydrogenophilalia, Oligoflexia, and/or other classes) levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting select Proteobacteria levels in the second sample; and
comparing the levels of select Proteobacteria in the second gut microbiome sample to levels of select Proteobacteria in the first gut microbiome sample,
wherein if the select Proteobacteria levels in the second sample are higher than the select Proteobacteria levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating treatment of diarrhea in an individual at risk of developing or who has developed or who has IBS, said method comprising:
detecting Ruminococcus levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Ruminococcus levels in the second sample; and
comparing the levels of Ruminococcus in the second gut microbiome sample to the levels of Ruminococcus in the first gut microbiome sample,
wherein if the Ruminococcus levels in the second sample are higher than the Ruminococcus levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating treatment of belching in an individual at risk of developing or who has developed or who has IBS, said method comprising:
detecting select Bacteroidaceae levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting select Bacteroidaceae levels in the second sample; and
comparing the levels of select Bacteroidaceae in the second gut microbiome sample to the levels of select Bacteroidaceae in the first gut microbiome sample,
wherein if the select Bacteroidaceae levels in the second sample are lower than the select Bacteroidaceae levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating treatment of overall well-being in an individual at risk of developing or who has developed or who has IBS, said method comprising:
detecting Anaerostipes and/or Mogibacterium levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Anaerostipes and/or Mogibacterium levels in the second sample; and
comparing the levels of Anaerostipes and/or Mogibacterium in the second gut microbiome sample to levels of Anaerostipes and/or Mogibacterium in the first gut microbiome sample,
wherein if the Anaerostipes and/or Mogibacterium levels in the second sample are lower than the Anaerostipes and/or Mogibacterium levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating treatment for bloating in an individual at risk of developing or who has developed or who has IBS, said method comprising:

Detecting baseline levels of at least one gut microbiome bacteria selected from the group consisting of Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, and Eggerthella in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting treatment levels of the at least one gut microbiome bacteria in the second sample;
determining a second measurement of the IBS-related parameter of the individual at the second time point;
comparing the baseline levels of the at least one gut microbiome bacteria to the treatment levels of said at least one gut microbiome bacteria, and
comparing the first measurement of the IBS-related parameter and the second measurement of the IBS-related parameter,
wherein if the Haemophilus, Pasteurellaceae, Enterobacteriaceae, Gammaproteobacteria, Granulicatella, Lachnospira, Oribacterium, and/or select Fusobacteriaceae treatment levels are lower than the baseline levels, and/or the Alistipes, Parabacteroides, Subdoligranulum, and/or Eggerthella treatment levels are higher than the baseline levels and the second IBS-related parameter is improved compared to the first IBS-related parameter, continuing the dosage regimen for the individual.

It is of note that while it may be more convenient to obtain gut microbiome samples and IBS-related parameter measurements at the same time, this is not a requirement of the invention. That is, the samples do not necessarily need to be taken at exactly the same time, but may be taken separately within a reasonable time period and still be considered as having been taken at either the first time point or the second time point as the case may be. The means for storing suitable samples for measurement of bacterial levels are well known in the art.

The individual who is at risk of developing IBS may be at risk based on genetic predisposition, familial history, heredity, lifestyle and/or one or more IBS-related parameters being out of range, for example, elevated anxiety or incidence of depression. As discussed above, the individual may also be an individual who has IBS, that is, an individual who has been diagnosed with IBS. Similarly, the individual may be an individual who has developed IBS, that is, an individual who has recently developed IBS and who may or may not have been diagnosed with IBS.

According to another aspect of the invention, there is provided a method for determining efficacy of a microbiome modulating of constipation in an individual at risk of developing or who has developed or who has IBS, said method comprising:
detecting Gammaproteobacteria, Granulicatella, and/or Alistipes levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting Gammaproteobacteria, Granulicatella, and/or Alistipes levels in the second sample;
determining a second measurement of the IBS-related parameter of the individual at the second time point;
comparing the levels of a given bacterium/bacteria in the second gut microbiome sample to levels of those same bacterium/bacteria in the first gut microbiome sample, and
comparing the first measurement of the IBS-related parameter and the second measurement of the IBS-related parameter,
wherein if the Gammaproteobacteria and/or Granulicatella levels in the second sample are lower than the levels in the first sample and/or Alistipes levels in the second sample are higher than the levels in the first sample, and the second IBS-related parameter is improved compared to the first IBS-related parameter, continuing the dosage regimen for the individual.

It is of note that while it may be more convenient to obtain Gammaproteobacteria, Granulicatella, and/or Alistipes samples and IBS-related parameter measurements at the same time, this is not a requirement of the invention. That is, the samples do not necessarily need to be taken at exactly the same time, but may be taken separately within a reasonable time period and still be considered as having been taken at either the first time point or the second time point as the case may be. The means for storing suitable samples for measurement of bacterial levels are well known in the art.

The individual who is at risk of developing IBS may be at risk based on genetic predisposition, familial history, heredity, lifestyle and/or one or more IBS-related parameters being out of range, for example, elevated anxiety or incidence of depression. As discussed above, the individual may also be an individual who has IBS, that is, an individual who has been diagnosed with IBS. Similarly, the individual may be an individual who has developed IBS, that is, an individual who has recently developed IBS and who may or may not have been diagnosed with IBS.

According to another aspect of the invention, there is provided a method for determining efficacy of a microbiome modulating of abdominal pain in an individual at risk of developing or who has developed or who has IBS, said method comprising:
detecting select Proteobacteria (defined as belonging to classes Acidithiobacilia, Hydrogenophilalia, Oligoflexia, and/or other classes) levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting select Proteobacteria levels in the second sample;
determining a second measurement of the IBS-related parameter of the individual at the second time point;
comparing the levels of select Proteobacteria in the second gut microbiome sample to levels of select Proteobacteria in the first gut microbiome sample, and
comparing the first measurement of the IBS-related parameter and the second measurement of the IBS-related parameter, wherein if the select Proteobacteria levels in the second sample are higher than the select Proteobacteria
levels in the first sample, and the second IBS-related parameter is improved compared to the first IBS-related parameter, continuing the dosage regimen for the individual.

It is of note that while it may be more convenient to obtain select Proteobacteria samples and IBS-related parameter measurements at the same time, this is not a requirement of the invention. That is, the samples do not necessarily need to be taken at exactly the same time, but may be taken separately within a reasonable time period and still be considered as having been taken at either the first time point or the second time point as the case may be. The means for storing suitable samples for measurement of bacterial levels are well known in the art.

The individual who is at risk of developing IBS may be at risk based on genetic predisposition, familial history, heredity, lifestyle and/or one or more IBS-related parameters being out of range, for example, elevated anxiety or incidence of depression. As discussed above, the individual may also be an individual who has IBS, that is, an individual who has been diagnosed with IBS. Similarly, the individual may be an individual who has developed IBS, that is, an individual who has recently developed IBS and who may or may not have been diagnosed with IBS.

According to another aspect of the invention, there is provided a method for determining efficacy of a microbiome modulating of diarrhea in an individual at risk of developing or who has developed or who has IBS, said method comprising:
detecting Ruminococcus levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting Ruminococcus levels in the second sample;
determining a second measurement of the IBS-related parameter of the individual at the second time point;
comparing the levels of Ruminococcus in the second gut microbiome sample to levels of Ruminococcus in the first gut microbiome sample, and
comparing the first measurement of the IBS-related parameter and the second measurement of the IBS-related parameter,
wherein if the Ruminococcus levels in the second sample are higher than the Ruminococcus levels in the first sample, and the second IBS-related parameter is improved compared to the first IBS-related parameter, continuing the dosage regimen for the individual.

It is of note that while it may be more convenient to obtain Ruminococcus samples and IBS-related parameter measurements at the same time, this is not a requirement of the invention. That is, the samples do not necessarily need to be taken at exactly the same time, but may be taken separately within a reasonable time period and still be considered as having been taken at either the first time point or the second time point as the case may be. The means for storing suitable samples for measurement of bacterial levels are well known in the art.

The individual who is at risk of developing IBS may be at risk based on genetic predisposition, familial history, heredity, lifestyle and/or one or more IBS-related parameters being out of range, for example, elevated anxiety or incidence of depression. As discussed above, the individual may also be an individual who has IBS, that is, an individual who has been diagnosed with IBS. Similarly, the individual may be an individual who has developed IBS, that is, an individual who has recently developed IBS and who may or may not have been diagnosed with IBS.

According to another aspect of the invention, there is provided a method for determining efficacy of a microbiome modulating of belching in an individual at risk of developing or who has developed or who has IBS, said method comprising:
detecting select Bacteroidaceae levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting select Bacteroidaceae levels in the second sample;
determining a second measurement of the IBS-related parameter of the individual at the second time point;
comparing the levels of select Bacteroidaceae in the second gut microbiome sample to levels of select Bacteroidaceae in the first gut microbiome sample, and
comparing the first measurement of the IBS-related parameter and the second measurement of the IBS-related parameter,
wherein if the select Bacteroidaceae levels in the second sample are lower than the select Bacteroidaceae levels in the first sample, and the second IBS-related parameter is improved compared to the first IBS-related parameter, continuing the dosage regimen for the individual.

It is of note that while it may be more convenient to obtain select Bacteroidaceae samples and IBS-related parameter measurements at the same time, this is not a requirement of the invention. That is, the samples do not necessarily need to be taken at exactly the same time, but may be taken separately within a reasonable time period and still be considered as having been taken at either the first time point or the second time point as the case may be. The means for storing suitable samples for measurement of bacterial levels are well known in the art.

The individual who is at risk of developing IBS may be at risk based on genetic predisposition, familial history, heredity, lifestyle and/or one or more IBS-related parameters being out of range, for example, elevated anxiety or incidence of depression. As discussed above, the individual may also be an individual who has IBS, that is, an individual who has been diagnosed with IBS Similarly, the individual may be an individual who has developed IBS, that is, an individual who has recently developed IBS and who may or may not have been diagnosed with IBS.

According to another aspect of the invention, there is provided a method for determining efficacy of a microbiome modulating of overall well-being in an individual at risk of developing or who has developed or who has IBS, said method comprising:
detecting Anaerostipes and/or Mogibacterium levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting Anaerostipes and/or Mogibacterium levels in the second sample;
determining a second measurement of the IBS-related parameter of the individual at the second time point;
comparing the levels of Anaerostipes and/or Mogibacterium in the second gut microbiome sample to levels of Anaerostipes and/or Mogibacterium in the first gut microbiome sample, and
comparing the first measurement of the IBS-related parameter and the second measurement of the IBS-related parameter,
wherein if the Anaerostipes and/or Mogibacterium levels in the second sample are lower than the Anaerostipes and/or Mogibacterium levels in the first sample, and the second IBS-related parameter is improved compared to the first IBS-related parameter, continuing the dosage regimen for the individual.

It is of note that while it may be more convenient to obtain Anaerostipes and/or Mogibacterium samples and IBS-related parameter measurements at the same time, this is not a requirement of the invention. That is, the samples do not necessarily need to be taken at exactly the same time, but may be taken separately within a reasonable time period and still be considered as having been taken at either the first time point or the second time point as the case may be. The means for storing suitable samples for measurement of bacterial levels are well known in the art.

The individual who is at risk of developing IBS may be at risk based on genetic predisposition, familial history, heredity, lifestyle and/or one or more IBS-related parameters being out of range, for example, elevated anxiety or incidence of depression. As discussed above, the individual may also be an individual who has IBS, that is, an individual who has been diagnosed with IBS Similarly, the individual may be an individual who has developed IBS, that is, an individual who has recently developed IBS and who may or may not have been diagnosed with IBS.

According to an aspect of the invention, there is provided a method for determining efficacy of a gut microbiome modulating treatment for bloating in an individual at risk of developing or who has developed or who has IBS, said method comprising:

Detecting baseline levels of at least one gut microbiome bacteria selected from the group consisting of: Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, and Eggerthella in a first gut microbiome sample from the individual at a first time point;
administering to the individual a microbiome modulating treatment on a dosage regimen for a suitable period of time;
following the suitable period of time, obtaining a second gut microbiome sample from the individual;
detecting treatment levels of the at least one gut microbiome bacteria in the second sample; and
comparing the baseline levels of the at least one gut microbiome bacteria in the second gut microbiome sample to the treatment levels of said at least one gut microbiome bacteria,
wherein if the Haemophilus, Pasteurellaceae, Enterobacteriaceae, Gammaproteobacteria, Granulicatella, Lachnospira, Oribacterium, and/or select Fusobacteriaceae treatment levels are lower than the baseline levevls levels, and/or the Alistipes, Parabacteroides, Subdoligranulum, and/or Eggerthella treatment levels are higher than the baseline levels, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating treatment of constipation in an individual at risk of developing or who has developed or who has IBS, said method comprising:
detecting Gammaproteobacteria, Granulicatella, and/or Alistipes levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Gammaproteobacteria, Granulicatella, and/or Alistipes levels in the second sample; and
comparing the levels of a given bacterium/bacteria in the second gut microbiome sample to levels of those same bacterium/bacteria in the first gut microbiome sample,
wherein if the Gammaproteobacteria and/or Granulicatella levels in the second sample are lower than the levels in the first sample and/or Alistipes levels in the second sample are higher than the levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating treatment of abdominal pain in an individual at risk of developing or who has developed or who has IBS, said method comprising:
detecting select Proteobacteria (defined as belonging to classes Acidithiobacilia, Hydrogenophilalia, Oligoflexia, and/or other classes) levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting select Proteobacteria levels in the second sample; and
comparing the levels of select Proteobacteria in the second gut microbiome sample to levels of select Proteobacteria in the first gut microbiome sample,
wherein if the select Proteobacteria levels in the second sample are higher than the select Proteobacteria levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating treatment of diarrhea in an individual at risk of developing or who has developed or who has IBS, said method comprising:
detecting Ruminococcus levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Ruminococcus levels in the second sample; and
comparing the levels of Ruminococcus in the second gut microbiome sample to levels of Ruminococcus in the first gut microbiome sample,
wherein if the Ruminococcus levels in the second sample are higher than the Ruminococcus levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating treatment of belching in an individual at risk of developing or who has developed or who has IBS, said method comprising:
detecting select Bacteroidaceae levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting select Bacteroidaceae levels in the second sample; and
comparing the levels of select Bacteroidaceae in the second gut microbiome sample to levels of select Bacteroidaceae in the first gut microbiome sample,
wherein if the select Bacteroidaceae levels in the second sample are lower than the select Bacteroidaceae levels in the first sample, continuing the dosage regimen for the individual.

In some embodiments, at the first time point and the second time point, at least one IBS-related parameter of the individual is measured and these two measurements are also compared.

According to another aspect of the invention, there is provided a method for determining efficacy of a microbiome modulating treatment of overall well-being in an individual at risk of developing or who has developed or who has IBS, said method comprising:
detecting Anaerostipes and/or Mogibacterium levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Anaerostipes and/or Mogibacterium levels in the second sample; and
comparing the levels of Anaerostipes and/or Mogibacterium in the second gut microbiome sample to levels of Anaerostipes and/or Mogibacterium in the first gut microbiome sample,
wherein if the Anaerostipes and/or Mogibacterium levels in the second sample are lower than the Anaerostipes and/or Mogibacterium levels in the first sample, continuing the dosage regimen for the individual.

In some embodiments, at the first time point and the second time point, at least one IBS-related parameter of the individual is measured and these two measurements are also compared.

According to an aspect of the invention, there is provided a method for determining efficacy of a gut microbiome modulating treatment for bloating in an individual at risk of developing or who has developed or who has IBS, said method comprising:

Detecting baseline levels of at least one gut microbiome bacteria selected from the group consisting of Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, and Eggerthella in a first gut microbiome sample from the individual at a first time point;
administering to the individual a microbiome modulating treatment on a dosage regimen for a suitable period of time;
following the suitable period of time, obtaining a second gut microbiome sample from the individual;
detecting treatment levels of the at least one gut microbiome bacteria in the second sample; and
comparing the treatment levels of the at least one gut microbiome bacteria to the baseline levels of said at least one gut microbiome bacteria,
wherein if the Haemophilus, Pasteurellaceae, Enterobacteriaceae, Gammaproteobacteria, Granulicatella, Lachnospira, Oribacterium, and/or select Fusobacteriaceae treatment levels are lower than the baseline levels, and/or the Alistipes, Parabacteroides, Subdoligranulum, and/or Eggerthella treatment levels are higher than the baseline levels, the microbiome modulating treatment is effective. If this is the case, the treatment, that is, the dosage regimen, is continued.

According to another aspect of the invention, there is provided a method for determining efficacy of a microbiome modulating treatment of constipation in an individual at risk of developing or who has developed or who has IBS, said method comprising:
detecting Gammaproteobacteria, Granulicatella, and/or Alistipes levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Gammaproteobacteria, Granulicatella, and/or Alistipes levels in the second sample; and
comparing the levels of a given bacterium/bacteria in the second gut microbiome sample to levels of those same bacterium/bacteria in the first gut microbiome sample,
wherein if the Gammaproteobacteria and/or Granulicatella levels in the second sample are lower than the levels in the first sample and/or Alistipes levels in the second sample are higher than the levels in the first sample, the microbiome modulating treatment is effective. If this is the case, the treatment, that is, the dosage regimen, is continued.

According to another aspect of the invention, there is provided a method for determining efficacy of a microbiome modulating treatment of abdominal pain in an individual at risk of developing or who has developed or who has IBS, said method comprising:
detecting select Proteobacteria (defined as belonging to classes Acidithiobacilia, Hydrogenophilalia, Oligoflexia, and/or other classes) levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting select Proteobacteria levels in the second sample; and
comparing the levels of select Proteobacteria in the second gut microbiome sample to levels of select Proteobacteria in the first gut microbiome sample,
wherein if the select Proteobacteria levels in the second sample are higher than the select Proteobacteria levels in the first sample, the microbiome modulating treatment is effective. If this is the case, the treatment, that is, the dosage regimen, is continued.

According to another aspect of the invention, there is provided a method for determining efficacy of a microbiome modulating treatment of diarrhea in an individual at risk of developing or who has developed or who has IBS, said method comprising:
detecting Ruminococcus levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Ruminococcus levels in the second sample; and
comparing the levels of Ruminococcus in the second gut microbiome sample to levels of Ruminococcus in the first gut microbiome sample,
wherein if the Ruminococcus levels in the second sample are higher than the Ruminococcus levels in the first sample, the microbiome modulating treatment is effective. If this is the case, the treatment, that is, the dosage regimen, is continued.

According to another aspect of the invention, there is provided a method for determining efficacy of a microbiome modulating treatment of belching in an individual at risk of developing or who has developed or who has IBS, said method comprising:
detecting select Bacteroidaceae levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting select Bacteroidaceae levels in the second sample; and
comparing the levels of select Bacteroidaceae in the second gut microbiome sample to levels of select Bacteroidaceae in the first gut microbiome sample,
wherein if the select Bacteroidaceae levels in the second sample are lower than the select Bacteroidaceae levels in the first sample, the microbiome modulating treatment is effective. If this is the case, the treatment, that is, the dosage regimen, is continued.

According to another aspect of the invention, there is provided a method for determining efficacy of a microbiome modulating treatment of overall well-being in an individual at risk of developing or who has developed or who has IBS, said method comprising:
detecting Anaerostipes and/or Mogibacterium levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Anaerostipes and/or Mogibacterium levels in the second sample; and
comparing the levels of Anaerostipes and/or Mogibacterium in the second gut microbiome sample to levels of AnAnaerostipes and/or Mogibacterium in the first gut microbiome sample,
wherein if the Anaerostipes and/or Mogibacterium levels in the second sample are lower than the Anaerostipes and/or Mogibacterium levels in the first sample the microbiome modulating treatment is effective. If this is the case, the treatment, that is, the dosage regimen, is continued.

According to another aspect of the invention, there is provided a method for determining efficacy of a microbiome modulating treatment for bloating in an individual at risk of developing or who has developed or who has IBS, said method comprising:
detecting baseline levels of at least one gut microbiome bacteria selected from the group consisting of Haemophilus, Pasteurellaceae, Enterobacteriaceae, Gammaproteobacteria, Granulicatella, Lachnospira, Oribacterium, select Fusobacteriaceae, Alistipes, Parabacteroides, Subdoligranulum, and Eggerthella in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting treatment levels of the at least one gut microbiome bacteria in the second sample;
determining a second measurement of the IBS-related parameter of the individual at the second time point;
comparing the treatment levels of the at least one gut microbiome bacteria to the baseline levels of said at least one gut microbiome bacteria, and
comparing the first measurement of the IBS-related parameter and the second measurement of the IBS-related parameter,
wherein if the Haemophilus, Pasteurellaceae, Enterobacteriaceae, Gammaproteobacteria, Granulicatella, Lachnospira, Oribacterium, and/or select Fusobacteriaceae treament levels are lower than the baseline levels, and/or the Alistipes, Parabacteroides, Subdoligranulum, and/or Eggerthella treatment levels are higher than the baseline levels and the second IBS-related parameter is improved compared to the first IBS-related parameter, the gut microbiome modulating treatment is effective. If that is the case, then the treatment, that is, the dosage regimen, is continued.

It is of note that while it may be more convenient to obtain gut microbiome samples and IBS-related parameter measurements at the same time, this is not a requirement of the invention. That is, the samples do not necessarily need to be taken at exactly the same time, but may be taken separately within a reasonable time period and still be considered as having been taken at either the first time point or the second time point as the case may be. The means for storing suitable samples for measurement of bacterial levels are well known in the art.

The individual who is at risk of developing IBS may be at risk based on genetic predisposition, familial history, heredity, lifestyle and/or one or more IBS-related parameters being out of range, for example, elevated anxiety or incidence of depression. As discussed above, the individual may also be an individual who has IBS, that is, an individual who has been diagnosed with IBS Similarly, the individual may be an individual who has developed IBS, that is, an individual who has recently developed IBS and who may or may not have been diagnosed with IBS.

According to another aspect of the invention, there is provided a method for determining efficacy of a microbiome modulating of constipation in an individual at risk of developing or who has developed or who has IBS, said method comprising:
detecting Gammaproteobacteria, Granulicatella, and/or Alistipes levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting Gammaproteobacteria, Granulicatella, and/or Alistipes levels in the second sample;
determining a second measurement of the IBS-related parameter of the individual at the second time point;
comparing the levels of a given bacterium/bacteria in the second gut microbiome sample to levels of those same bacterium/bacteria in the first gut microbiome sample, and
comparing the first measurement of the IBS-related parameter and the second measurement of the IBS-related parameter,
wherein if the Gammaproteobacteria and/or Granulicatella levels in the second sample are lower than the levels in the first sample and/or Alistipes levels in the second sample are higher than the levels in the first sample, and the second IBS-related parameter is improved compared to the first IBS-related parameter, the gut microbiome modulating treatment is effective. If that is the case, then the treatment, that is, the dosage regimen, is continued.

It is of note that while it may be more convenient to obtain Gammaproteobacteria, Granulicatella, and/or Alistipes samples and IBS-related parameter measurements at the same time, this is not a requirement of the invention. That is, the samples do not necessarily need to be taken at exactly the same time, but may be taken separately within a reasonable time period and still be considered as having been taken at either the first time point or the second time point as the case may be. The means for storing suitable samples for measurement of bacterial levels are well known in the art.

The individual who is at risk of developing IBS may be at risk based on genetic predisposition, familial history, heredity, lifestyle and/or one or more IBS-related parameters being out of range, for example, elevated anxiety or incidence of depression. As discussed above, the individual may also be an individual who has IBS, that is, an individual who has been diagnosed with IBS. Similarly, the individual may be an individual who has developed IBS, that is, an individual who has recently developed IBS and who may or may not have been diagnosed with IBS.

According to another aspect of the invention, there is provided a method for determining efficacy of a microbiome modulating of abdominal pain in an individual at risk of developing or who has developed or who has IBS, said method comprising:
detecting select Proteobacteria (defined as belonging to classes Acidithiobacilia, Hydrogenophilalia, Oligoflexia, and/or other classes) levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting select Proteobacteria levels in the second sample;
determining a second measurement of the IBS-related parameter of the individual at the second time point;
comparing the levels of select Proteobacteria in the second gut microbiome sample to levels of select Proteobacteria in the first gut microbiome sample, and
comparing the first measurement of the IBS-related parameter and the second measurement of the IBS-related parameter,
wherein if the select Proteobacteria levels in the second sample are higher than the select Proteobacteria levels in the first sample, and the second IBS-related parameter is improved compared to the first IBS-related parameter, the gut microbiome modulating treatment is effective. If that is the case, then the treatment, that is, the dosage regimen, is continued.

It is of note that while it may be more convenient to obtain select Proteobacteria samples and IBS-related parameter measurements at the same time, this is not a requirement of the invention. That is, the samples do not necessarily need to be taken at exactly the same time, but may be taken separately within a reasonable time period and still be considered as having been taken at either the first time point or the second time point as the case may be. The means for storing suitable samples for measurement of bacterial levels are well known in the art.

The individual who is at risk of developing IBS may be at risk based on genetic predisposition, familial history, heredity, lifestyle and/or one or more IBS-related parameters being out of range, for example, elevated anxiety or incidence of depression. As discussed above, the individual may also be an individual who has IBS, that is, an individual who has been diagnosed with IBS. Similarly, the individual may be an individual who has developed IBS, that is, an individual who has recently developed IBS and who may or may not have been diagnosed with IBS.

According to another aspect of the invention, there is provided a method for determining efficacy of a microbiome modulating of diarrhea in an individual at risk of developing or who has developed or who has IBS, said method comprising:
detecting Ruminococcus levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting Ruminococcus levels in the second sample;
determining a second measurement of the IBS-related parameter of the individual at the second time point;
comparing the levels of Ruminococcus in the second gut microbiome sample to levels of Ruminococcus in the first gut microbiome sample, and
comparing the first measurement of the IBS-related parameter and the second measurement of the IBS-related parameter,
wherein if the Ruminococcus levels in the second sample are higher than the Ruminococcus levels in the first sample, and the second IBS-related parameter is improved compared to the first IBS-related parameter, the gut microbiome modulating treatment is effective. If that is the case, then the treatment, that is, the dosage regimen, is continued.

It is of note that while it may be more convenient to obtain Ruminococcus samples and IBS-related parameter measurements at the same time, this is not a requirement of the invention. That is, the samples do not necessarily need to be taken at exactly the same time, but may be taken separately within a reasonable time period and still be considered as having been taken at either the first time point or the second time point as the case may be. The means for storing suitable samples for measurement of bacterial levels are well known in the art.

The individual who is at risk of developing IBS may be at risk based on genetic predisposition, familial history, heredity, lifestyle and/or one or more IBS-related parameters being out of range, for example, elevated anxiety or incidence of depression. As discussed above, the individual may also be an individual who has IBS, that is, an individual who has been diagnosed with IBS Similarly, the individual may be an individual who has developed IBS, that is, an individual who has recently developed IBS and who may or may not have been diagnosed with IBS.

According to another aspect of the invention, there is provided a method for determining efficacy of a microbiome modulating of belching in an individual at risk of developing or who has developed or who has IBS, said method comprising:
detecting select Bacteroidaceae levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting select Bacteroidaceae levels in the second sample;
determining a second measurement of the IBS-related parameter of the individual at the second time point;
comparing the levels of select Bacteroidaceae in the second gut microbiome sample to levels of select Bacteroidaceae in the first gut microbiome sample, and
comparing the first measurement of the IBS-related parameter and the second measurement of the IBS-related parameter,
wherein if the select Bacteroidaceae levels in the second sample are lower than the select Bacteroidaceae levels in the first sample, and the second IBS-related parameter is improved compared to the first IBS-related parameter, the gut microbiome modulating treatment is effective. If that is the case, then the treatment, that is, the dosage regimen, is continued.

It is of note that while it may be more convenient to obtain select Bacteroidaceae samples and IBS-related parameter measurements at the same time, this is not a requirement of the invention. That is, the samples do not necessarily need to be taken at exactly the same time, but may be taken separately within a reasonable time period and still be considered as having been taken at either the first time point or the second time point as the case may be. The means for storing suitable samples for measurement of bacterial levels are well known in the art.

The individual who is at risk of developing IBS may be at risk based on genetic predisposition, familial history, heredity, lifestyle and/or one or more IBS-related parameters being out of range, for example, elevated anxiety or incidence of depression. As discussed above, the individual may also be an individual who has IBS, that is, an individual who has been diagnosed with IBS. Similarly, the individual may be an individual who has developed IBS, that is, an individual who has recently developed IBS and who may or may not have been diagnosed with IBS.

According to another aspect of the invention, there is provided a method for determining efficacy of a microbiome modulating of overall well-being in an individual at risk of developing or who has developed or who has IBS, said method comprising:
detecting Anaerostipes and/or Mogibacterium levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting Anaerostipes and/or Mogibacterium levels in the second sample;
determining a second measurement of the IBS-related parameter of the individual at the second time point;
comparing the levels of Anaerostipes and/or Mogibacterium in the second gut microbiome sample to levels of Anaerostipes and/or Mogibacterium in the first gut microbiome sample, and
comparing the first measurement of the IBS-related parameter and the second measurement of the IBS-related parameter,
wherein if the Anaerostipes and/or Mogibacterium levels in the second sample are lower than the Anaerostipes and/or Mogibacterium levels in the first sample, and the second IBS-related parameter is improved compared to the first IBS-related parameter, the gut microbiome modulating treatment is effective. If that is the case, then the treatment, that is, the dosage regimen, is continued.

It is of note that while it may be more convenient to obtain Anaerostipes and/or Mogibacterium samples and IBS-related parameter measurements at the same time, this is not a requirement of the invention. That is, the samples do not necessarily need to be taken at exactly the same time, but may be taken separately within a reasonable time period and still be considered as having been taken at either the first time point or the second time point as the case may be. The means for storing suitable samples for measurement of bacterial levels are well known in the art.

The individual who is at risk of developing IBS may be at risk based on genetic predisposition, familial history, heredity, lifestyle and/or one or more IBS-related parameters being out of range, for example, elevated anxiety or incidence of depression. As discussed above, the individual may also be an individual who has IBS, that is, an individual who has been diagnosed with IBS. Similarly, the individual may be an individual who has developed IBS, that is, an individual who has recently developed IBS and who may or may not have been diagnosed with IBS.

As discussed herein, we demonstrate a method for detecting and treating individuals with IBS-related symptoms who are sensitive to microbiome-targeted therapeutic intervention using a microbiome modulating compound. In some embodiments, the microbiome modulating compound is prebiotic resistant potato starch.

In other embodiments, the microbiome modulating compound is selected from the group consisting of: resistant potato starch, probiotic genera, species, and strains; prebiotics supporting growth of probiotic genera, species and strains; resistant starch from corn, tapioca, banana, grains, tubers and the like; fructooligosaccharides; galactooligosaccharides; xylooligosaccharides; mannanoligosaccharides; arabinoxylooligosaccharides; arabinogalactan polysaccharides; and galactomannan polysaccharides.

Dietary changes that support the growth of healthy bacteria, including the probiotic bacteria listed above:
Dietary treatments that increase the availability of microbiota-accessible carbohydrates (MACs), for example prebiotics, to select Proteobacteria, Alistipes, Parabacteroides, Subdoligranulum, Eggerthella, and/or Ruminococcus, including those prebiotics listed above.
Dietary treatments that reduce the availability of protein and/or peptides and/or amino acids and/or other fermentation substrates to Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium in the digestive tract.
Antibiotics that target Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium or another bacterium/other bacteria that facilitate the growth of Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium.

The probiotic genera, species and strains may be selected from the group consisting of: Bifidobacterium; Staphylococcus; Clostridium; Lactobacilli; Prevotella; Barnsiella; Parasutterella; and combinations thereof;

The resistant starch may be RS1, RS2, RS3, RS4, or RS5.

The corn may be high amylose maize.

The grains may be barley, wheat, sorghum, oats or the like.

Examples of suitable fructooligosaccharides include but are by no means limited to inulin and inulin-type fructans.

The galactooligosaccharides may be of varying lengths, for example, between 2 and 8 saccharide units, and may include various linkages of galactose for example but by no means limited to β-(1-4), β-(1-6) galactose, and a terminal glucose.

The Xylooligosaccharides may be composed of xylose or related C5 sugar oligosaccharides.

The mannanoligosaccharides, may be for example glucomannanoligosaccharides.

Suitable galactomannan polysaccharides include guar gum.

In other embodiments, the microbiome modulating compound is selected from the group consisting of: resistant potato starch, probiotic genera, species, and strains; prebiotics supporting growth of probiotic genera, species and strains; resistant starch from corn, tapioca, banana, grains, tubers and the like; fructooligosaccharides; galactooligosaccharides; xylooligosaccharides; mannanoligosaccharides; arabinoxylooligosaccharides; arabinogalactan polysaccharides; galactomannan polysaccharides; dietary changes that support the growth of probiotic bacteria; dietary treatments that reduce the availability of protein and/or peptides and/or amino acids and/or other fermentation substrates to Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium in the digestive tract; dietary treatments that increase the availability of microbiota-accessible carbohydrates (MACS) and/or prebiotics and/or other fermentation substrates to select Proteobacteria, Alistipes, Parabacteroides, Subdoligranulum, Eggerthella, and/or Ruminococcus in the digestive tract; and antibiotics that target Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium or another bacterium/other bacteria that facilitate the growth of Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium.

In yet other embodiments, the microbiome modulating compound is selected from the group consisting of: resistant potato starch, probiotic genera, species, and strains; prebiotics supporting growth of probiotic genera, species and strains; resistant starch from corn, tapioca, banana, grains, tubers and the like; fructooligosaccharides; galactooligosaccharides; xylooligosaccharides; mannanoligosaccharides; arabinoxylooligosaccharides; arabinogalactan polysaccharides; galactomannan polysaccharides; dietary changes that support the growth of probiotic bacteria; dietary treatments that reduce the availability of protein and/or peptides and/or amino acids and/or other fermentation substrates to Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium in the digestive tract; antibiotics that target Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium or another bacterium/other bacteria that facilitate the growth of Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium; mixed plant cell wall fibers; beta-glucans; resistant dextrins; resistant maltodextrins; limit dextrins; polydextrose; alginate; pectin polysaccharides; hydroxypropylmethylcellulose; chitin; chondroitin-containing compounds; and glucosamine-containing compounds.

Preferably, the mixed plant cell wall fibers comprise two or more of the following plant cell wall fibers in varying proportions: cellulose, pectin, lignin, beta-glucan, and arabinoxylan regardless of source.

The Beta-glucans may be from cereal, such as for example, mixed-link (1-3, 1-4) beta-glucans from oat, barley, rye, wheat, or the like, or from fungal sources, for example, yeast, mushroom, and the like.

Resistant dextrins, resistant maltodextrins, and limit dextrins may be from wheat, corn, or other suitable sources. These non-digestible oligosaccharides of glucose molecules are joined by digestible linkages and non-digestible α-1,2 and α-1,3 linkages.

The polydextrose may be highly branched and may contain α- and β-1-2, 1-3, 1-4 and 1-6 linkages, with the 1-6 linkage predominating in the polymer.

The alginate may be β-1,4-D-mannuronic acid and α-1,4-L-guluronic acid organized in homopolymeric compounds of either mannuronate or guluronate, or as heteropolymeric compounds, expressed as mannuronic acid to guluronic acid ratio.

The pectin polysaccharides may have a backbone chain of α-(1→4)-linked D-galacturonic acid units interrupted by the insertion of (1→2)-linked L-rhamnopyranosyl residues in adjacent or alternate positions. These compounds are present in cell walls and intracellular tissues of fruits, vegetables, legumes, and nuts.

Hydroxypropylmethylcellulose, also known as Hypromellose, is a propylene glycol ether of methylcellulose containing methoxyl groups and hydroxypropyl group.

The chitin may be from for example from fungi or arthropods.

Suitable chondroitin-containing compounds includes chondroitin sulfate from animal sources.

Suitable glucosamine-containing compounds includes glucosamine sulfate from animal sources.

In some embodiments, the gut microbiome modulating treatment may be or may also include spores from a single strain or specie of bacteria, yeast, or other fungi; bacteriophage or a combination of bacteriophages; or an exogenously produced metabolite or metabolites normally derived from the metabolism of the gut microbiome, also known as postbiotics or parabiotics.

As will be appreciated by one of skill in the art, an IBS-related parameter as used herein refers to a parameter that is associated with or measured as part of monitoring the symptoms of IBS.

In some embodiments of the invention, the IBS-related parameter is selected from the group consisting of: Bristol Stool Chart or other bowel movement quality scores; personal diaries scoring bowel movement frequency, bloating, abdominal pain, gas, belching, and/or overall well-being; reports of bowel movement frequency, bloating, abdominal pain, gas, belching, and/or overall well-being made to a health care practitioner, such as a gastroenterologist, general practitioner, dietitian, nutritionist, psychologist, or psychiatrist; digital applications recording bowel movement frequency, bloating, abdominal pain, gas, belching, and/or overall well-being.

As will be appreciated by one of skill in the art, in some embodiments, the IBS-related parameter selected may be associated with or considered informative of one or more of the specific IBS symptoms being treated with the microbiome modulating compound. For example, the Bristol Stool Chart could be the IBS-related parameter assessed if the symptom being treated or monitored for improvement was either constipation or diarrhea.

As will be appreciated by one of skill in the art, other means for monitoring or measuring improvement in or quantifying IBS-related symptoms are known in the art and can be used within the invention for determining IBS-related parameters.

As discussed herein, the individual may suffer from: abdominal pain, including bloating or abdominal distension, approximately one or more days per week; increases or decreases in pain with defecation; pain associated with changes in stool frequency; and/or pain associated with changes in stool shape. As will be appreciated by one of skill in the art, an “improvement” in one or more of these symptoms may be associated with for example, a reduction in frequency, a reduction in severity or a reduction in associated pain. That is, the symptoms have “improved” in that the individual suffers from these symptoms either less frequently and/or to a lesser degree.

In another embodiment of the invention, the individual is at risk of developing IBS due to family history, lifestyle factors, or due to co-morbidities such as anxiety or depression.

The period of time, that is, the suitable period of time may be for example about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks or longer. As will be appreciated by one of skill in the art, a “dosage regimen” will comprise taking an effective amount of the treatment for the duration of the suitable period of time, as discussed herein. That is, as will be appreciated by one of skill in the art, the “suitable period of time” is typically a period of time that is long enough for an individual capable of being treated, that is, capable of having the severity of one or more symptoms associated with IBS reduced compared prior to beginning administration, to notice a difference in their symptomology or for changes in the gut microbiome, as described herein, to be detected. It is further noted that during the suitable period of time of the dosage regimen, the microbiome modulating compound is administered at “an effective amount”, as described herein.

Levels of bacteria may be measured using any suitable means known in the art. For example, levels of these bacteria may be measured using real-time polymerase chain reaction (RT-PCR)-based methods; qualitative PCR (qPCR) based methods; by microbiome sequencing directed at any sequence that defines the bacteria to the genus, species, and/or strain level, including but not limited to the 16S V4 ribosomal subunit sequence; by shotgun metagenomic sequencing; by quantitative fluorescent in situ hybridization (FISH) with probes recognizing sequence that defines the bacteria of interest, including but not limited to the 16S V4 ribosomal subunit sequence; or by antibody or cell-binding based methods.

As will be appreciated by one of skill in the art, the bacterial levels are being measured over time. Consequently, levels of bacteria may be determined by direct measurement, using suitable means known in the art, for example, such as those discussed above. Alternatively, the level of bacteria of interest in a given sample may be compared to an internal control, for example, using the abundance of Bacteroides or other common commensal unrelated to IBS symptomology as the reference value. As will be apparent to one of skill in the art, Bacteroides is both common (found in most gut microbiomes) and abundant (making up a large proportion of each gut microbiome), and accordingly is suitable to be used as an internal control. However, other suitable candidates for use as an internal control will be readily apparent to one of skill in the art. Alternatively, the control may be a non-biological control. Furthermore, as will be appreciated by one of skill in the art, the control does not necessarily need to be repeated with each measurement.

As will be apparent to those of skill in the art, an “effective amount” of a gut microbiome modulating compound is an amount that is believed to be sufficient to reduce Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium levels, and/or increase select Proteobacteria, Alistipes, Parabacteroides, Subdoligranulum, Eggerthella, and/or Ruminococcus, and improve at least one IBS-related parameter in the individual when administered on a dosage regimen or schedule over the suitable period of time. Such an effective amount will of course depend on the specific gut microbiome modulating compound being administered as well as other factors such as the age, weight, general condition and severity of symptoms of the individual.

It is respectfully noted that with knowledge of the link between for example select Fusobacteriaceae levels as defined herein and IBS-related symptoms, one of skill in the art could develop other methods for detecting changes in for example “select Fusobacteriaceae” levels, for example, by including or excluding other members of Fusobacteriaceae in the determination of “select Fusobacteriaceae” levels and then determining the effect of the inclusion or exclusion of said one or more members of Fusobacteriaceae in said measurement.

For example, ‘select Fusobacteriaceae’ is based on taxonomic assignment to identities present in the Greengenes 13-8-99 database, which excludes Cetobacterium, Fusobacterium, Propionigenium, Psychrilyobacter, and u144 to assign bacteria to the other Fusobacteriaceae category, but the database does not facilitate assignment to other genera belonging to Fusobacteriaceae, including Hypnocyclicus or Ilyobacter. One of skill in the art could, as an example, further refine the connection between IBS-related symptoms and ‘select Fusobacteriaceae’ to include the genus Hypnocyclicus but exclude Ilyobacter based on additional genomic detail.

Similarly, one of skill in the art may further refine the connection between IBS-related symptoms and ‘select Bacteroidaceae’ to include or exclude the genera Acetofilamentum, Acetothermus, Desulfoarculus, Massilibacteroides, or Phocaeicola, which are not present in the Greengenes β-8-99 database, based on additional genomic detail.

That is, as discussed above, these “select” or “other” bacteria represent “unidentifiable” or “unidentified” bacteria as discussed herein. As such, as used herein, it is noted that “unidentified” or “unidentifiable” will be understood by one of skill in the art as bacteria strains not present in the Greengenes 13-8-99 database. It is further noted that elimination of other strains from “unidentified” can be carried out using additional taxonomic information and/or other means of identification and is considered to be within routine experimentation by one of skill in the art.

As discussed herein, the prebiotic microbiome therapeutic may be resistant potato starch, delivered daily or as needed, for as long as the IBS-related markers continue to show improvement compared to baseline levels, that is, compared to IBS-related markers taken or measured prior to the start of administration.

As discussed herein, the effective amount of resistant potato starch may be for example 2 to 40 g, 2 to 30 g, 2 to 20 g, 5 to 40 g, 5 to 30 g, 5 g to 20 g, or 10 to 20 g of resistant potato starch.

The effective amount may be administered in one or more doses during the day.

As used herein, “daily” does not necessarily mean “every day” but may mean 9 out 10 days; 8 out of 9 days; 7 out of 8 days; 6 out of 7 days; 5 out of 6 days; 4 out of 5 days; 3 out of 4 days; 2 out of 3 days; 1 out of 2 days or combinations thereof.

The heterogeneous nature of IBS has made it difficult to establish definitive diagnostic criteria but this has not prevented investigations seeking a ‘microbiome signature’ for IBS. Here, we explored the relationships between changes in the microbiome and changes in IBS-related symptoms in healthy people consuming prebiotic resistant potato starch (RPS). Consistent with other findings, we cannot detect a signature of dysbiosis that captures all symptoms of IBS. Rather, our correlation analysis revealed discrete relationships between different bacteria and IBS-related symptoms, suggesting not only that IBS-related symptom-bacterium relationships (ie. ‘fingerprints’) exist but that RPS can modulate many bacteria to provide relief from IBS-related symptoms. Those with a given ‘fingerprint’ can be identified by gut microbiome analysis and their symptoms treated with RPS.

Our findings indicate a role for various member of phylum Proteobacteria in IBS symptoms. Consistent with this, elevated levels of some Proteobacteria (Krogius-Kurikka et al. 2009. BMC Gastroenterology), especially the class Gammaproteobacteria, family Pasteurellaceae, including genus Haemophilus (Saulnier et al. 2011. Gastroenterology; Veiga et al. 2014. Sci Rep), have been reported in patients with IBS. The abundance of members of class Gammaproteobacteria, including family Enterobacteriaceae and genus Haemophilus, have been reported to be elevated in IBS patients and were positively correlated with IBS symptom scores (Rajilic-Stojanovic et al. 2011. Gastroenterology). Thus, the resolution of bloating and constipation combined with reductions in members of Gammaproteobacteria observed in participants consuming RPS are consistent the role of these bacteria in people with IBS.

Other members of Proteobacteria have also been associated with IBS, including Pseudomonas (class Gammaproteobacteria, order Pseudomonadales; Kerckhoffs et al. 2011. J Med Microbiol) and Parasutterella (class Betaproteobacteria, order Burkholderiales; Chen et al. 2018. J Gastroenterol Hepatol). We identified correlations between abdominal pain and both an unclassified member of Proteobacteria and Oxalobacteraceae (class Betaproteobacteria). In both cases, increasing levels of these Proteobacteria were associated with resolutions in abdominal pain, and were discrete from those Proteobacteria associated with bloating and constipation, highlighting the disparate roles Proteobacteria play in the human gut.

Similar to Gammaproteobacteria, levels of Granulicatella (phylum Firmicutes) and Alistipes (phylum Bacteroidetes) were correlated with both bloating and constipation, further highlighting the connection between these symptoms. Both Granulicatella and Haemophilus are heavily bound by ileal IgA antibodies in patients with IBS-D (Liu et al. 2020. Clin Transl Gastroenterol), indicating active responses against these genera during IBS pathogenesis, and Granulicatella is more abundant in individuals with IBS (Zhu et al. 2019. Front Cell Infect Microbiol), which is consistent with reductions in Granulicatella being correlated with reductions in constipation and bloating.

Several species of Alistipes have been associated with increased pain in pediatric IBS patients, including Alistipes putredinis (Saulnier et al. 2011. Gastroenterology) and unclassified Alistipes were more abundant in people with Myalgic encephalomyelitis/chronic fatigue syndrome coinciding with IBS (Nagy-Szakal et al. 2017. Microbiome). Alistipes levels increase in response to anti-constipation medication Lubiprostone in mice (Mulch et al. 2013. Dig Dis Sci), while levels were lower in children with functional constipation compared to controls (de Meij et al. 2016. PLoS ONE). Oligosaccharide treatment in mice improved constipation measures via increased water content and decreased transit time, but led to reduced levels of Alistipes (Wang et al. 2017A. Food Funct) as did probiotic Bifidobacterium treatment of loperamide-induced constipated mice (Wang et al. 2017B. Food Funct). Given these varied findings, it is possible that Alistipes roles in IBS-related symptoms, such as the reduction of bloating and constipation, depend on dietary inputs like RPS.

Alistipes and Parabacteroides are both members of order Bacteroidales, and increasing levels of Parabacteroides were correlated with decreased bloating in people consuming RPS. Abundance of Parabacteroides has previously been shown to distinguish healthy controls from IBS patients (Noor et al. 2010. BMC Gastroenterol) and levels of these bacteria are depleted in patients with genetic (Henstrom et al. 2018. Gut) and conventionally diagnosed forms of IBS (Zhu et al. 2019. Front Cell Infect Microbiol). While prebiotics have been shown to enhance the abundance of Parabacteroides in vitro (Carlson et al. 2016. Anaerobe), increasing levels have not previously been connected to any IBS symptom resolution, suggesting that RPS may hold advantages over other prebiotics.

In addition to Granulicatella, three other genera from phylum Firmicutes were correlated with changes in bloating. Decreasing levels of Lachnospira and Oribacterium (family Lachnospiraceae) were associated with improvements in bloating while increasing levels of Subdoligranulum (family Oscillospiraceae) were associated with bloating improvement. Lachnospira and Lachnospiraceae members are more abundant in those with IBS compared to healthy controls (Zhu et al. 2019. Front Cell Infect Microbiol), consistent with our observations that reducing levels of these bacteria improves IBS-related symptoms. Similarly, dietary intervention with a low fermentable substrate diet led to improvements in IBS symptoms and increases in Subdoligranulum (Chumpitazi et al. 2014. Gut Microbes) and levels of Subdoligranulum were depleted in patients with IBS-D (Liu et al. 2020. BMC Microbiol). Therefore, increasing levels of these bacteria via RPS intervention is consistent with other reports of IBS symptom improvement.

Decreasing levels of two other genera from order Eubacteriales, Anaerostipes and Mogibacterium, were correlated with increased reports of well-being. Anaerostipes levels have been correlated with bad mood (Li et al. 2016. Neurogastroenterol Motil) but showed no association with quality of life scores in response to inulin supplementation (Vandeputte et al. 2017. Gut). Relative abundance of Anaerostipes was significantly lower in pediatric Crohn's disease patients with higher levels of perceived stress (Mackner et al. 2020. Psychoneuroendocrinology). Anaerostipes levels were relatively lower in people with obsessive compulsive disorder (Turna et al. 2020. Acta Psychiatr Scand) and was one of several depleted genera in multiple psychiatric diseases (Li et al. 2020. J Psychiatr Res). It is therefore unclear how changing Anaerostipes or Mogibacterium levels are related to feelings of well-being.

Similar to fellow family Oscillospiraceae member Subdoligranulum, RPS consumption that increased levels of Ruminococcus was beneficial, leading to lower levels of diarrhea. Differing types of Ruminococcus have been associated with different IBS disease states (Lyra et al. 2009. World J Gastroenterol; Malinen et al. 2010. World J Gastroenterol; Saulnier et al. 2011. Gastroenterology, Rajilic-Stojanovic et al. 2011. Gastroenterology, Shukla et al. 2015. Dig Dis Sci, Hynonen et al. 2016. Anaerobe, Mazzawi et al. 2018. PLoS ONE). However, Ruminococcus levels did not differentiate between IBS-C and IBS-D participants (Shukla et al. 2015. Dig Dis Sci). Probiotic intervention with Lactobacillus paracasei CNCM 1-1572 led to Ruminococcus reductions in IBS patients without symptomatic improvement (Cremon et al. 2018. United European Gastroenterol J). Feedlot cattle with hemorrhagic diarrhea have significantly lower levels of Ruminoccus compared to healthy controls (Zeineldin et al. 2018. Microb Pathog), as do piglets with diarrhea (Yang et al. 2019. Microbiologyopen), and Ruminococcus levels can be used to predict the development of diarrhea in veal calves (Ma et al. 2020. ISME J). Treatment of piglets with Gegen Qinlian decoction, a Chinese herbal formulation, increased Ruminococcus while decreasing diarrhea (Liu et al. 2019. Front Microbiol) and Ruminococcus bromii is one of a handful of recognized resistant starch degrading bacteria (Ze et al. 2012. ISME J). Taken together, these reports are generally consistent with a beneficial role for Ruminococcus in IBS symptom mitigation in people consuming RPS.

Not all genera identified in our study have previously been linked to IBS. There are no reported associations between Oribacterium and IBS, although Oribacterium belongs to family Lachnospiraceae, which was significantly more abundant in patients with IBS compared to controls (Zhu et al. 2019. Front Cell Infect Microbiol). Similarly, Eggerthella, Fusobacteriaceae, and cyanobacteria have not been associated with IBS symptoms. Changes in Granulicatella have not been associated with constipation nor have changes in Mogibacterium been associated with well-being. Neither Bacteroidaceae nor Dehalobacterium have been associated with burping or belching. Victivallis has not previously been associated with reports of overall well-being but levels have been shown to increase in response to high amylose maize starch supplementation in people (Zhang et al. 2019. Sci Rep).

Thus, as discussed above, gut microbiome dysbiosis contributes to symptomology in IBS. Changes in the levels of Haemophilus, Pasteurellaceae, Enterobacteriaceae, Gammaproteobacteria, Granulicatella, Lachnospira, Oribacterium, select Fusobacteriaceae, select Proteobacteria, select Bacteroidaceae, Anaerostipes Mogibacterium, Alistipes, Parabacteroides, Subdoligranulum, Eggerthella, and/or Ruminococcus serve as markers for changes in the microbiome-mediated influence on IBS symptoms. Specifically, it is believed that levels of Haemophilus, Pasteurellaceae, Enterobacteriaceae, Gammaproteobacteria, Granulicatella, Lachnospira, Oribacterium, select Fusobacteriaceae, select Proteobacteria, select Bacteroidaceae, Anaerostipes, Mogibacterium, Alistipes, Parabacteroides, Subdoligranulum, Eggerthella, and/or Ruminococcus serve as a marker of IBS-related parameters but it is unclear to what extent each genus is a driver of microbiome-mediated IBS symptom burden or relief. Accordingly, monitoring levels of these bacteria in combination with at least one IBS-related parameter provides information on the effectiveness of gut microbiome related treatments. If Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes, and/or Mogibacterium levels decrease in combination with improvements in one or more of the IBS-related parameters, this indicates that the individual can be treated using gut microbiome-based treatments. Similarly, if select Proteobacteria, Alistipes, Parabacteroides, Subdoligranulum, Eggerthella, and/or Ruminococcus levels increase in combination with improvements in one or more of the IBS-related parameters, this also indicates that the individual can be treated using gut microbiome-based treatments.

Alternatively, if Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes, and/or Mogibacterium levels decrease but the IBS-related parameters do not improve, or if select Proteobacteria, Alistipes, Parabacteroides, Subdoligranulum, Eggerthella, and/or Ruminococcus levels increase and the IBS-related parameters do not improve, the IBS symptoms may be more heavily influenced by other factors, for example, genetic predisposition, diet, activity levels or the like and the gut microbiome modulating treatment should be stopped and replaced with more conventional treatments for IBS-related symptoms.

In summary, screening for Haemophilus, Pasteurellaceae, Enterobacteriaceae, Gammaproteobacteria, Granulicatella, Lachnospira, Oribacterium, select Fusobacteriaceae, select Proteobacteria, select Bacteroidaceae, Anaerostipes Mogibacterium, Alistipes, Parabacteroides, Subdoligranulum, Eggerthella, and/or Ruminococcus levels in combination with IBS-related parameters will identify those individuals who will benefit from positive modulation of the gut microbiome. The effectiveness of this strategy can then be measured by monitoring levels of these bacteria in combination with IBS-related measures. Our findings support these statements for the following reasons: 1) Changes in bacteria and changes in IBS-related symptoms were correlated at either multiple time points and/or multiple doses of prebiotic supplementation, demonstrating consistency, 2) Changes in bacteria and changes in IBS-related symptoms were documented in the placebo group were only relevant if the correlation was enhanced by prebiotic supplementation, so bacteria found to fluctuate with IBS-related symptom severity were only included if their correlation could be leveraged using prebiotic supplementation, and 3) the discrete correlations between IBS-related symptoms and various bacteria does not support a generalizable dysbiosis in IBS, thereby necessitating microbiome monitoring the judge the efficacy to microbiome interventions that target these genera. In other words, our data suggest that different IBS-related symptoms are related to ‘fingerprints’ of IBS dysbiosis and that microbiome modulating treatments that target these fingerprints can improve the important symptoms in an individualized manner.

This screen holds several advantages over methods focused on modifying the gut microbiota as a means of improving IBS-related symptoms. First, our data demonstrate that combinations of bacteria-symptom correlations exist and can be exploited by prebiotic supplementation. Unlike previous studies, which attempted to characterize IBS dysbiosis by comparing IBS gut microbiomes to those in healthy controls, our data identify numerous functional relationships (ie. ‘fingerprints’) between bacteria in the gut microbiome and IBS symptoms, facilitating tailored symptom resolution strategies for this incredibly heterogeneous disorder. The ability to observe changes in both symptoms and levels of bacteria in response to an intervention overcomes the limitations of observational studies where the relationship between gut bacteria and symptoms could not be elucidated.

Second, the use of IBS-related symptom correlations identified outside of an IBS diagnosis mean that utility of these findings is not limited to people with IBS. This is especially beneficial in the case of IBS, where the diagnosis of individuals likely leads to the exclusion of affected individuals due to subjective and imprecise diagnostic criteria, including the gold-standard Rome IV criteria. For example, a person presenting with abdominal pain associated with changes in stool frequency and consistency would not meet IBS diagnostic criteria unless they suffered from such events at least once a week on average over the course of 3 months (Palsson et al. 2016. Gastroenterology). However, such a person would clearly stand to benefit from the diagnostic and intervention methods described above. The utility for these methods therefore extends to anyone with IBS-related symptoms.

Finally, the use of Pearson correlation coefficients, which determine linear proportionality, is particularly helpful because it allows the proportional improvement in IBS-related parameters to be inferred from the changes in abundance of bacteria in the gut microbiome. These results suggest that for as long as these bacteria are detectable (ie. A non-zero value), changes in the abundance of those bacteria will be informative for the health outcome of the host. Practically speaking, this means that the screen is predictive regardless of absolute levels, be they minimum or maximum values. This provides a generic method by which to test the efficacy of microbiome-based therapies for improving IBS-related symptoms.

### Materials and Methods

### Investigational Product

The resistant starch (RS) used in this study was MSPrebiotic (MSPrebiotics Inc., Carberry, MB), an unmodified resistant potato starch (RS type 2) with an RS content of 60% (AOAC 2002.02). MSPrebiotic has been previously described (Alfa et al. 2018. Front Med, Alfa et al. 2018. Clin Nutr). The placebo used was fully digestible corn starch (Amioca; Ingredion, Brampton, ON) and contains no RS. Nutrasource (Guelph, ON) provided randomization services for the clinical trial supplies. These services were carried out by personnel not involved in the collection of study data to ensure blinding of the study.

### Clinical Trial Structure, per Protocol Determination, and Sample Collection

This study was conducted at Nutrasource in Guelph, ON, Canada, a Clinical Research Unit that recruited participants from the general population in Guelph, ON and surrounding area. Onsite monitoring was conducted by Nutrasource according to the Clinical Monitoring Plan. The data management and statistical analyses for this study were provided by Nutrasource contract research organization and were conducted according to Nutrasource's Standard Operating Procedures based on International Council for Harmonization (ICH), Health Canada Natural Health Product Regulations, and the Food and Drug Administration (FDA) regulations and guidance documents.

The study protocol and other related documents (e.g., Informed Consent Form, Study Diaries, etc.) were approved by Canadian Shield Ethics Review Board on 29 Oct. 2019. This study was conducted in accordance with the protocol and with the consensus ethical principles derived from international guidelines, including the Declaration of Helsinki and Council for International Organizations of Medical Sciences International Ethical Guidelines, applicable ICH Good Clinical Practice (GCP) guidelines, and applicable local and federal laws and regulations.

The Investigator or their representative explained the nature of the study to the participant or their legally authorized representative and answered all questions regarding the study. Participants were informed that their participation was voluntary. If participants wished to participate in the study, they or their legally authorized representative were required to sign a statement of informed consent that met the requirements of local regulations, ICH guidelines, and the REB or study center. The source document included a statement that written informed consent was obtained before the participant was enrolled in the study and the date the written consent was obtained. The authorized person obtaining the informed consent also signed the informed consent form (ICF). A copy of the ICF was provided to the participant or the participant's legally authorized representative.

Participants enrolled in this study were generally healthy adult males and females between 18-69 years of age (inclusive) with a BMI of 18.0 and ≤34.9 kg/m² (inclusive). Included participants agreed to not use any new vitamin, mineral or dietary supplement product until after the study completion and to not take any vitamins, minerals or dietary supplements 14 days prior to Visit 2 (Randomization) until the completion of Visit 4, since consumption of these products may bias the results for the test product. Individuals with a BMI over 34.9 kg/m² were excluded as their health and any related metabolic changes may impact the results of this study. For similar health reasons, any individuals with a diagnosis of irritable bowel syndrome, dyspepsia, significant gastrointestinal disorders or other major diseases were excluded. A formal sample size calculation was not performed. The sample size of 25 subjects per study group is an industry suggested minimum number to investigate changes in the primary endpoint (changes in the microbiome).

This was a randomized, double-blind, placebo-controlled 3-arm parallel group study. A total of 98 participants were screened for eligibility to obtain the required sample size of 75 participants (25 participants per study arm), who were enrolled in the study. The identity of the study intervention was blinded to the study staff and participants. The study included a pre-screening visit, a screening visit from 30 days up to 14 days prior to randomization, a run in-phase of 14 to 17 days prior to randomization, a baseline visit (Day 0) during which the randomization was performed, and 2 subsequent study visits at Weeks 1 and 4, respectively. During the screening visit, bowel habit diary, food records, and stool collection instructions and materials (including 2 fecal sample collection containers) were provided. During the run-in period, the participants recorded their daily bowel habits for 14 to 17 days. They also completed their food records 3 days prior to the first fecal sample collection and again, for 3 days prior to each subsequent stool collection. As fecal sample collection was spontaneous, participants who produced a fecal sample that was collected prior to the 3rd day of following the same food intake was documented but this was not considered a protocol deviation.

Participants collected fecal samples in 2 sample collection containers (one sample for molecular analysis and short chain fatty acid analysis and one sample for metabolomics analysis) within 72 hours prior to Day 0 and transferred it to the clinic site within 24 hours of collection. Stool samples were collected in OMNIgene-Gut kits (DNA Genotek, Ottawa, ON), which stabilizes the microbiome DNA. During the baseline visit (Day 0), the participants were randomized to receive 1 of the 3 study interventions i.e., MSPrebiotic® high dose (7 g resistant potato starch), MSPrebiotic® low dose (3.5 g resistant potato starch plus 3.5 g digestible corn starch), or placebo (7 g digestible corn starch) as indicated by the randomization scheme. The first dose of study intervention was administered and new food records, bowel habit/daily diaries, stool collection supplies, a copy of their previous food records, and a 31-day supply of the study intervention was provided to the study participants.

At Visit 3 (Week 1), previous food records, bowel habit diary, and unused study interventions/empty packaging were collected, and compliance was calculated. New food records, bowel habit/daily diaries, a copy of their previous food records, and stool collection supplies were provided to the participants.

At Visit 4 (Week 4, the final study visit), previous food records, bowel habit diary, and unused study products/empty packaging were collected, and compliance was calculated. The collected fecal samples were analyzed for DNA sequencing of 16S RNA gene and SCFA and one of the fecal samples is stored for possible future metabolomics analysis. Participants collected fecal samples within 72 hours prior to Visit 3 (Week 1) and Visit 4 (Week 4) and transferred them to the clinic site within 24 hours of each collection. Microbiome sequencing was directed at the 16s rRNA V4 region (Microbiome Insights, Vancouver, BC).

Participants were instructed to return to their next visits (i.e., V3 and V4) with all sachets they were sent home with and to not throw away any sachets (open or unopened). The study product was returned at V3 and re-dispensed after compliance was calculated. Documentation of compliance was calculated based on the amount of study product consumed compared to the total amount of study product expected to have been consumed for the given duration. Compliance was considered acceptable if an average of at least 80% of the assigned study product for the study period was consumed. One participant per treatment arm discontinued the study before Visit 4, and 2 participants in MSPrebiotic® high dose arm were excluded from the per protocol set due to non-compliance (<80% overall compliance and use of prohibited drugs, respectively).

### Microbiome Analysis

16Sv4 amplicons generated from fecal samples collected in OMNIgene-Gut kits (DNA Genotek) were sequenced on a MiSeq platform (Illumina, San Diego, CA). MiSeq-generated Fastq files were quality-filtered and clustered into 97% similarity operational taxonomic units (OTUs) using the mothur software package [http://www.mothur.org]. The resulting dataset had 59086 OTUs (including those occurring once with a count of 1, or singletons). An average of 30860 quality-filtered reads were generated per sample. Sequencing quality for R1 and R2 was determined using FastQC 0.11.5. Bacteria levels indicated are the relative abundance.

### IBS-Related Symptom Scoring

Participants rated their level of bloating, abdominal pain, gas, belching, and overall well-being daily throughout the trial on a scale from 0 (none) to 3 (severe). Values were averaged during the run-in period (˜14 days prior to intervention; Baseline) as well as during the first week (Week 1) and last week (Week 4) of the intervention period.

During the same time periods, participants score their bowel movements using the Bristol Stool Chart (BSC), where Type 1=constipation with hard, round stools and Type 7=watery diarrhea. It was important to score constipation and diarrhea separately because some individuals suffer from both symptoms, and these symptoms could be incorrectly normalized or lost if BSC scores were simply averaged over the study interval. To this end, constipation scores were derived from bowel movements with BSC Types 1-4, where Type 1 was scored 4, Type 2 scored 3, and Types 3 and 4 were each scored 1. Diarrhea scores were derived from bowel movements with BSC Types 3-7, where Type 7 was scored 8, Type 6 scored 7, Type 5 scored 6, and Types 3 and 4 were each scored 1. Diarrhea stools received higher numerical values than those for constipation due to the urgency typically associated with diarrhea. Values were averaged separately for constipation and diarrhea during the run-in period (˜14 days prior to intervention; Baseline), during the first week (Week 1), and during the last week (Week 4) of the intervention period.

### Statistical Analysis

Changes in IBS-related symptoms, including diarrhea, constipation, bloating, abdominal pain, gas, belching, and overall well-being, were determined by subtracting Baseline values from Week 1 or Week 4 values. Changes in relative abundance for each bacteria category were similarly calculated. Pearson correlation coefficients were calculated between changes in symptom and changes in relative abundance for each symptom-bacteria pair among the 7 g/day, 3.5 g/day, and placebo treatment arms. In total, 6,006 correlations between changes in IBS-related symptoms and changes in the gut microbiota were calculated and p values determined. P values presented are uncorrected. To control for multiple testing, symptom-bacteria correlations were considered significant only if they were directionally shared 1) between time points at 7 g/day and/or 2) between doses with p<0.05 for the first and p<0.1 for the second correlation. Using this method, the probability of Type 1 error is 0.005.

### REFERENCES

Alfa, M J, Strang, D, Tappia, P S, Graham, M, Van Domselaar, G, Forbes, J D, Laminman, V, Olson, N, DeGagne, P, Bray, D, Murray, B L, Dufault, B, Lix, L M. 2018. Clin Nutr. 37(3):797-807.
Alfa, M J, Strang, D, Tappia, PS, Olson, N, DeGagne, P, Bray, D, Murray, B L, Hiebert, B. 2018. Front Med (Lausanne). 4:260.
Carlson, J, Gould, T, Slavin, J. 2016. Anaerobe. 42:60-66.
Chen, Y-J, Wu, H, Wu, S-D, Lu, N, Wang, Y-T, Liu, H-N, Dong, L, Liu T-T, Shen, X-Z. 2018. J Gastroenterol Hepatol. 33(11):1844-1852.
Chumpitazi, B P, Hollister, E B, Oezguen, N, Tsai, C M, McMeans, A R, Luna, R A, Savidge, T C, Versalovic, J, Shulman, R J. 2014. Gut Microbes. 5(2):165-175.
Cremon, C, Guglielmetti, S, Gargari, G, Taverniti, V, Castellazzi, A M, Valsecchi, C, Tagliacarne, C, Fiore, W, Bellini, M, Bertani, L, Gambaccini, D, Cicala, M, Germanà, B, Vecchi, M, Pagano, I, Barbaro, M R, Bellacosa, L, Stanghellini, V, Barbara, G. 2018. United European Gastroenterol J. 6(4):604-613.
de Meij, T G J, de Groot, E F J, Eck, A, Budding, A E, Kneepkens, C M F, Benninga, M A, van Bodegraven, A A, Savelkoul, P H M. 2016. PLoS ONE. 11(10):e0164731.
Henström, H, Diekmann, L, Bonfiglio, F, Hadizadeh, F, Kuech, E-M, von Köckritz-Blickwede, M, Thingholm, L B, Zheng, T, Assadi, G, Dierks, C, Heine, M, Philipp, U, Distl, O, Money, M E, Belheouane, M, Heinsen, F-A, Rafter, J, Nardone, G, Cuomo, R, Usai-Satta, P, Galeazzi, F, Neri, M, Walter, S, Simrén, M, Karling, P, Ohlsson, B, Schmidt, P T, Lindberg, G, Dlugosz, A, Agreus, L, Andreasson, A , Mayer, E, Baines, J F, Engstrand, L, Portincasa, P, Bellini, M, Stanghellini, V, Barbara, G, Chang, L, Camilleri, M, Franke, A, Naim, H Y, D'Amato, M. 2018. Gut. 67(2):263-270.
Hynönen, U, Rasinkangas, P, Satokari, R, Paulin, L, de Vos, W M, Pietilä, TE, Kant, R, Palva, A. 2016. Anaerobe. 39:60-67.
Kerckhoffs, A P, Ben-Amor, K, Samsom, M, van der Rest, M E, de Vogel, J, Knol, J, Akkermans, L M A. 2011. J Med Microbiol. 60(Pt 2):236-245.
Krogius-Kurikka, L, Lyra, A, Malinen, E, Aarnikunnas, J, Tuimala, J, Paulin, L, Mäkivuokko, H, Kajander, K, Palva, A. 2009. BMC Gastroenterology. 9:95.
Li, L, Su, Q, Xie, B, Duan. L, Zhao, W, Hu, D, Wu, R, Liu, H. 2016. Neurogastroenterol Motil. 28(8):1233-1240.
Li, J, Ma, Y, Bao, Z, Gui, X, Li, A N, Yang, Z, Li, M D. 2020. J Psychiatr Res. 130:48-56.
Liu, C S, Liang, X, Wei, X H, Jin, Z, Chen, FL , Tang, Q F, Tan, X M. 2019. Front Microbiol. 10:825.
Liu, Y, Yuan, X, Li, L, Lin, L, Zuo, X, Cong, Y, Li, Y. 2020. Clin Transl Gastroenterol. 11(3):e00146.
Liu, T, Gu, X, Li, L-X, Li, M, Li, B, Cui, X, Zuo, X-L. 2020. BMC Microbiol. 20(1):168.
Lyra, A, Rinttilä, T, Nikkilä, J, Krogius-Kurikka, L, Kajander, K, Malinen, E, Mättö, J, Mäkelä, L, Palva, A. 2009. World J Gastroenterol. 15(47):5936-5945.
Ma, T, Villot, C, Renaud, D, Skidmore, A, Chevaux, E, Steele, M, Guan, L L. 2020. ISME J. 14(9):2223-2235.
Mackner, L M, Hatzakis, E, Allen, J M, Davies, R H, Kim, S C, Maltz, R M, Bailey, M T. 2020. Psychoneuroendocrinology. 111:104469.
Malinen, E, Krogius-Kurikka, L, Lyra, A, Nikkilä, J, Jääskeläinen, A, Rinttilä, T, Vilpponen-Salmela, T, von Wright, A J, Palva, A. 2010. World J Gastroenterol. 16(36):4532-4540.
Mazzawi, T, Lied, G A, Sangnes, D A, El-Salhy, M, Hov, J R, Gilja, O H, Hatlebakk, J G, Hausken, T. 2018. PLoS ONE. 13(11):e0194904.
Musch, M W, Wang, Y, Claud, E C, Chang, E B. 2013. Dig Dis Sci. 58(3):668-677.
Nagy-Szakal, D, Williams, B L, Mishra, N, Che, X, Lee, B, Bateman, L, Klimas, N G, Komaroff, A L, Levine, S, Montoya, J G, Peterson, D L, Ramanan, D, Jain, K, Eddy, M L, Hornig, M, Lipkin, W I. 2017. Microbiome. 5(1):44.
Noor, S O, Ridgway, K, Scovell, L, Kemsley, E K, Lund, E K, Jamieson, C, Johnson, I T, Narbad, A. 2010. BMC Gastroenterol. 10:134.
Palsson, O S, Whitehead, W E, Van Tilburg, M A L, Chang, L, Chey, W, Crowell, M D, Keefer, L, Lembo, A J, Parkman, H P, Rao, S S C, Sperber, A, Spiegel, B, Tack, J, Vanner, S, Walker, L S, Whorwell, P, Yang, Y. 2016. Gastroenterology, 150(6):1481-1491.
Pittayanon, R, Lau, J T, Yuan, Y, Leontiadis, G I, Tse, F, Surette, M, Moayyedi, P. 2019. Gastroenterology. 157(1):97-108.
Rajiliĉ-Stojanović, M, Biagi, E, Heilig, H G H J, Kajander, K, Kekkonen, R A, Tims, S, de Vos, W M. 2011. Gastroenterology. 141(5):1792-1801.
Saulnier, D M, Riehle, K, Mistretta, T-A, Diaz, M A, Mandal, D, Raza, S, Weidler, E M, Qin, X, Coarfa, C, Milosavljevic, A, Petrosino, J F, Highlander, S, Gibbs, R, Lynch, S V, Shulman, R J, Versalovic, J. 2011. Gastroenterology. 141(5):1782-1791.
Shukla, R, Ghoshal, U, Dhole, T N, Ghoshal, U C. 2015. Dig Dis Sci. 60(10):2953-2962.
Turna, J, Grosman Kaplan, K, Anglin, R, Patterson, B, Soreni, N, Bercik, P, Surette, M G, Van Ameringen, M. 2020. Acta Psychiatr Scand. doi: 10.1111/a cps.13175.
Vandeputte, D, Falony, G, Vieira-Silva, S, Wang, J, Sailer, M, Theis, S, Verbeke, K, Raes, J. 2017. Gut. 66(11):1968-1974.
Veiga P, Pons, N, Agrawal, A, Oozeer, R, Guyonnet, D, Brazeilles, R, Faurie, J-M, van Hylckama Vlieg, J E T, Houghton, L A, Whorwell P J, Ehrlich, S , Kennedy S P. 2014. Sci Rep. 4:6328.
Wang, L, Hu, L, Yan, S, Jiang, T, Fang, S, Wang, G, Zhao, Z, Zhang, H, Chen, W. 2017 (A). Food Funct. 8(5):1966-1978.
Wang, W, Hu, L, Xu, Q, Jiang, T, Fang, S, Wang, G, Zhao, J, Zhang, H, Chen, W. 2017 (B). Food Funct. 8(10):3587-3600.
Yang, Q, Huang, X, Wang, P, Yan, Z, Sun, W, Zhao, S, Gun, S. 2019. Microbiologyopen. 8(12):e923.
Ze, X, Duncan, S H, Louis, P, Flint, H J. 2012. ISME J. 6(8):1535-1543.
Zeineldin, M, Aldridge, B, Lowe, J. 2018. Microb Pathog. 115:123-130.
Zhang, L, Ouyang, Y, Li, H, Shen, L, Ni, Y, Fang, Q, Wu, G, Qian, L, Xiao, Y, Zhang, J, Yin, P, Panagiotou, G, Xu, G, Ye, J, Jia, W. 2019. Sci Rep. 9(1):4736.
Zhu, S, Liu, S, Li, H, Zhang, Z, Zhang, Q, Chen, L, Zhao, Y, Chen, Y, Gu, J, Min, L, Zhang S. 2019. Front Cell Infect Microbiol. 9:346.

TABLE 1
RPS consumption led to reductions in bloating associated with reductions in Gammaproteobacteria,
including Enterobacteriaceae, Pasteurellaceae, and Haemophilus, increases in Bacteroidales genera
Alistipes and Parabacteroides, and decreases in Lachnospiraeae genera Lachnospira and Oribacterium.

| | | | | | | Correlation | |
|---|---|---|---|---|---|---|---|
| Phylum | Class | Order | Family | Genus | p values | Type | Inference |
| Proteobacteria | Gamma- | — | — | Gamma- | 0.00207; | RPS effective | Decreasing |
| | proteobacteria | | | proteobacteria_ | 0.001439; | regardless of | Gammaproteobacteria |
| | | | | unclassified | 0.053566 | dose or duration | decreases bloating |
| | | Enterobacterales | Enterobacteriaceae | Enterobacteriaceae_ | 0.00288; | RPS effective at | Decreasing |
| | | | | unclassified | 0.023568 | higher dose | Enterobacteriaceae |
| | | | | | | | decreases bloating |
| | | Pasteurellales | Pasteurellaceae | Pasteurellaceae_ | 0.000983; | RPS effective at | Decreasing |
| | | | | unclassified | 0.024364 | higher dose | Pasteurellaceae |
| | | | | | | | decreases bloating |
| | | | | Haemophilus | 0.001086; | RPS effective at | Decreasing |
| | | | | | 0.023851 | higher dose | Haemophilus |
| | | | | | | | decreases bloating |
| Bacteroidetes | Bacteroidia | Bacteroidales | Rikenellaceae | Alistipes | 0.00761; | RPS effective at | Increasing Alistipes |
| | | | | | 0.08737 | higher dose | decreases bloating |
| | | | Tannerellaceae | Parabacteroides | 0.015156; | RPS effective | Increasing |
| | | | | | 0.022117; | regardless of | Parabacteroides |
| | | | | | 0.008832 | dose or duration | decreases bloating |
| Firmicutes | Bacilli | ? | ? | RFN20 | 0.077047; | Placebo effect | Placebo decreases |
| | | | | | 0.009968 | | RFN20 while |
| | | | | | | | increasing bloating |
| | | Lactobacillies | Carnobacteriaceae | Granulicatella | 0.026028; | RPS effective at | Decreasing |
| | | | | | 0.000954 | higher dose | Granulicatella |
| | | | | | | | decreases bloating |
| | Clostridia | Eubacteriales | Lachnospiraceae | Lachnospira | 0.001322; | RPS effective at | Decreasing |
| | | | | | 0.016224 | higher dose | Lachnospira |
| | | | | | | | decreases bloating |
| | | | | Oribacterium | 0.01443; | RPS effective at | Decreasing |
| | | | | | 0.024188 | higher dose | Oribacterium |
| | | | | | | | decreases boating |
| | | | Oscillospiraceae | Subdoligranulum | 0.015522; | RPS effective at | Increasing |
| | | | | | 0.06785 | higher dose | Subdoligranulum |
| | | | | | | | decreases bloating |
| Actinobacteria | Coriobacteriia | Eggerthellales | Eggerthellaceae | Eggerthella | 0.004383; | RPS effective | Increasing |
| | | | | | 0.046934; | regardless of | Eggerthella |
| | | | | | 0.011537 | dose or duration | decreases bloating |
| Fusobacteria | Fusobacteriia | Fusobacteriales | Fusobacteriaceae | Fusobacteriaceae_ | 0.001108; | RPS effective at | Decreasing |
| | | | | unclassified | 0.024188 | higher dose | Fusobacteriaceae |
| | | | | | | | decreases bloating |

TABLE 2
RPS reduced constipation by decreasing Gammaproteobacteria and Granulicatella, while increasing Alistipes.

| | | | | | | Correlation | |
|---|---|---|---|---|---|---|---|
| Phylum | Class | Order | Family | Genus | p values | Type | Inference |
| Proteobacteria | Gammaproteo- | — | — | Gammaproteobacteria_ | 0.000596; | RPS | Decreasing |
| | bacteria | | | unclassified | 0.040478; | effective | Gammaproteobacteria |
| | | | | | 0.045645 | regardless | decreases constipation |
| | | | | | | of dose or | |
| | | | | | | duration | |
| Firmicutes | Bacilli | Lactobacillies | Carnobacteriaceae | Granulicatella | 0.000555; | RPS | Decreasing |
| | | | | | 0.010586 | effective at | Granulicatella |
| | | | | | | higher dose | decreases constipation |
| Bacteroidetes | Bacteroidia | Bacteroidales | Rikenellaceae | Alistipes | 0.04573; | RPS | Increasing Alistipes |
| | | | | | 0.016206 | effective at | decreases constipation |
| | | | | | | longer | |
| | | | | | | duration | |

TABLE 3
Increases in Proteobacteria, including Oxalobacteraceae, led to reductions in abdominal pain,
while only changes in unclassified Proteobacteria were associated with RPS consumption.

| | | | | | | Correlation | |
|---|---|---|---|---|---|---|---|
| Phylum | Class | Order | Family | Genus | p values | Type | Inference |
| Proteobacteria | — | — | — | Proteobacteria_ | 0.049139; | RPS effective | Increasing |
| | | | | unclassified | 0.003914 | at higher dose | Proteobacteria |
| | | | | | | | decreases abdominal |
| | | | | | | | pain |
| | Betaproteo- | Burkholderiales | Oxalobacteraceae | Oxalobacteraceae_ | 0.051162; | Effect due to | Increasing |
| | bacteria | | | unclassified | 0.007377 | Placebo | Oxalobacteraceae |
| | | | | | | | decreases abdominal |
| | | | | | | | pain |

TABLE 4
Independent of RPS administration, changes in gas levels were proportional to changes in
Cyanobacteria levels

| Phylum | Class | Order | Family | Genus | p values | Correlation Type | Inference |
|---|---|---|---|---|---|---|---|
| Cyanobacteria | — | — | — | Cyanobacteria_ | 0.000123; | Independent of | Increasing |
| | | | | unclassified | 0.053718 | intervention, more | cyanobacteria |
| | | | | | | significant in placebo | increases gas |

TABLE 5
Changes in diarrhea levels were proportional to changes in Ruminococcus levels, but
correlations in RPS groups were more significant.

| Phylum | Class | Order | Family | Genus | p values | Correlation Type | Inference |
|---|---|---|---|---|---|---|---|
| Firmicutes | Clostridia | Eubacteriales | Oscillospiraceae | Ruminococcus | 0.053922; | Independent of | Increasing |
| | | | | | 0.059346; | intervention, though | Ruminococcus |
| | | | | | 0.005722; | stronger in RPS | decreases diarrhea |
| | | | | | 0.049757 | | |

TABLE 6
Independent of RPS administration, changes in belching were proportional to changes in
Dehalobacterium, while RPS reduced belching while reducing Bacteroidaceae levels.

| | | | | | | Correlation | |
|---|---|---|---|---|---|---|---|
| Phylum | Class | Order | Family | Genus | p values | Type | Inference |
| Firmicutes | Clostridia | Eubacteriales | Peptococcaceae | Dehalobacterium | 0.092659; | Effect due to | Decreasing |
| | | | | | 0.003033 | Placebo | Dehalobacterium |
| | | | | | | | increases belching |
| Bacteroidetes | Bacteroidia | Bacteroidales | Bacteroidaceae | Bacteroidaceae_ | 0.03765; | RPS effective | Decreasing |
| | | | | unclassified | 0.016553 | at higher dose | Bacteroidiaceae |
| | | | | | | | decreases belching |

TABLE 7
Placebo decreased Victivallis to reduce well-being, while improvements in well-being were more
significantly associated with decreases in Anaerostipes and Mogibacterium in RPS groups.

| Phylum | Class | Order | Family | Genus | p values | Correlation Type | Inference |
|---|---|---|---|---|---|---|---|
| Lentisphaerae | Lentisphaeria | Victivallales | Victivallaceae | Victivallis | 0.065197; | Effect due to Placebo | Decreasing |
| | | | | | 0.010276 | | Victivallis |
| | | | | | | | decreases well-being |
| Firmicutes | Clostridia | Eubacteriales | Lachnospiraceae | Anaerostipes | 0.032518; | Independent of | Decreasing |
| | | | | | 0.009058; | intervention, though | Anaerostipes |
| | | | | | 0.028789 | stronger in RPS | increases well-being |
| | | | | Mogibacterium | 0.08482; | Independent of | Decreasing |
| | | | | | 0.007906; | intervention, though | Mogibacterium |
| | | | | | 0.022809 | stronger in RPS | increases well-being |

TABLE 8
Significant correlations between changes in higher taxonomic groups and IBS symptoms.

| | Taxonomic | IBS-Related | | | | |
|---|---|---|---|---|---|---|
| Group | Level | Symptom | P values | Treatment | Duration | Inference |
| Proteobacteria | Phylum | Abdominal | 0.489501 | Placebo | 1 week | |
| | | Pain | 0.317763 | Placebo | 4 weeks | |
| | | | 0.834612 | 3.5 g | 1 week | |
| | | | 0.760181 | 3.5 g | 4 weeks | |
| | | | 0.014235 | 7 g | 1 week | RPS drives increases in Proteobacteria to transiently |
| | | | | | | reduce abdominal pain at high doses |
| | | | 0.760181 | 7 g | 4 weeks | |
| Gammaproteo- | Class | Bloating | 0.317809 | Placebo | 1 week | |
| bacteria | | | 0.336055 | Placebo | 4 weeks | |
| | | | 0.361234 | 3.5 g | 1 week | |
| | | | 0.718192 | 3.5 g | 4 weeks | |
| | | | 0.001122 | 7 g | 1 week | RPS drives decreases in Gammaproteobacteria to |
| | | | 0.019819 | 7 g | 4 weeks | reduce bloating at high doses |
| | | Constipation | 0.202418 | Placebo | 1 week | |
| | | | 0.164036 | Placebo | 4 weeks | |
| | | | 0.226064 | 3.5 g | 1 week | |
| | | | 0.121702 | 3.5 g | 4 weeks | |
| | | | 0.040504 | 7 g | 1 week | RPS drives decreases in Gammaproteobacteria to |
| | | | 0.027409 | 7 g | 4 weeks | reduce constipation at high doses |
| Enterobacteriaceae | Family | Bloating | 0.295642 | Placebo | 1 week | |
| | | | 0.879362 | Placebo | 4 weeks | |
| | | | 0.341471 | 3.5 g | 1 week | |
| | | | 0.917145 | 3.5 g | 4 weeks | |
| | | | 0.002574 | 7 g | 1 week | RPS drives decreases in Enterobacteriaceae to reduce |
| | | | 0.025374 | 7 g | 4 weeks | bloating at high doses |
| Pasteurellaceae | Family | Bloating | 0.220421 | Placebo | 1 week | |
| | | | 0.580465 | Placebo | 4 weeks | |
| | | | 0.974769 | 3.5 g | 1 week | |
| | | | 0.93063 | 3.5 g | 4 weeks | |
| | | | 0.001071 | 7 g | 1 week | RPS drives decreases in Pasteurellaceae to reduce |
| | | | 0.023895 | 7 g | 4 weeks | bloating at high doses |
| Fusobacteriaceae | Family | Bloating | 0.931367 | Placebo | 1 week | |
| | | | 0.801113 | Placebo | 4 weeks | |
| | | | 0.6685 | 3.5 g | 1 week | |
| | | | 0.999186 | 3.5 g | 4 weeks | |
| | | | 0.001404 | 7 g | 1 week | RPS drives decreases in Fusobacteriaceae to transiently |
| | | | | | | reduce bloating at high doses |
| | | | 0.230672 | 7 g | 4 weeks | |
| Bacteroidaceae | Family | Belching | 0.90688 | Placebo | 1 week | |
| | | | 0.235399 | Placebo | 4 weeks | |
| | | | 0.606389 | 3.5 g | 1 week | |
| | | | 0.899163 | 3.5 g | 4 weeks | |
| | | | 0.050122 | 7 g | 1 week | |
| | | | 0.508857 | 7 g | 4 weeks | |

1-14. (canceled)

15. A method for determining efficacy of a microbiome modulating treatment for IBS-related symptoms in an individual at risk of developing IBS or who has developed IBS or who has IBS, said method comprising:
detecting baseline levels of at least one gut microbiome bacteria selected from the group consisting of: Haemophilus, Pasteurellaceae, Enterobacteriaceae, Gammaproteobacteria, Granulicatella, Lachnospira, Oribacterium, Fusobacteriaceae not belonging to the genera Cetobacterium, Fusobacterium, Propionigenium, Psychrilyobacter, or u114 (hereafter ‘select Fusobacteriaceae’), Bacteroidaceae not belonging to the genera 5-7N15, Bacteroides, or BF311 (hereafter ‘select Bacteroidaceae’), Anaerostipes and/or Mogibacterium in a first gut microbiome sample from the individual at a first time point;
administering to the individual a microbiome modulating treatment on a dosage regimen for a suitable period of time;
following the suitable period of time, obtaining a second gut microbiome sample from the individual;
detecting treatment levels of the at least one gut microbiome bacteria in the second sample; and
comparing the treatment levels of the at least one gut microbiome bacteria to the baseline levels of said at least one gut microbiome bacteria,
wherein if the treatment levels of Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium are lower than the baseline levels of Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium, continuing the dosage regimen for the individual.

16-21. (canceled)

22. The method according to claim 15 wherein the microbiome modulating compound is selected from the group consisting of:
resistant potato starch, probiotic genera, species, and strains; prebiotics supporting growth of probiotic genera, species and strains; resistant starch from corn, tapioca, banana, grains, tubers and the like; fructooligosaccharides; galactooligosaccharides; xylooligosaccharides; mannanoligosaccharides; arabinoxylooligosaccharides; arabinogalactan polysaccharides; galactomannan polysaccharides; dietary changes that support the growth of probiotic bacteria; dietary treatments that increase the availability of resistant starch and/or prebiotics and/or other fermentation substrates to bacteria that inhibit the growth of Haemophilus, Pasteurellaceae, Enterobacteriaceae, Gammaproteobacteria, Granulicatella, Lachnospira, Oribacterium, Fusobacteriaceae not belonging to the genera Cetobacterium, Fusobacterium, Propionigenium, Psychrilyobacter, or u114 (hereafter ‘select Fusobacteriaceae’), Bacteroidaceae not belonging to the genera 5-7N15, Bacteroides, or BF311 (hereafter ‘select Bacteroidaceae’), Anaerostipes and/or Mogibacterium in the digestive tract; and antibiotics that target Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium; mixed plant cell wall fibers; beta-glucans; resistant dextrins; resistant maltodextrins; limit dextrins; polydextrose; alginate; pectin polysaccharides; hydroxypropylmethylcellulose; chitin; chondroitin-containing compounds; and glucosamine-containing compounds.

23-24. (canceled)

25. The method according to claim 15 wherein the suitable period of time is from 1 week to 6 months.

26. (canceled)

27. The method according to claim 15 wherein the gut microbiome modulating compound is resistant potato starch.

28. The method according to claim 15 wherein the effective amount is 2 to 40 g per day of resistant potato starch.

29-91. (canceled)

92. A method for determining efficacy of a microbiome modulating treatment of constipation in an individual at risk of developing or who has developed or who has IBS, said method comprising:
detecting Gammaproteobacteria, Granulicatella, and/or Alistipes levels in a first gut microbiome sample from the individual at a first time point;
administering to the individual a microbiome modulating treatment on a dosage regimen for a suitable period of time;
following the suitable period of time, obtaining a second gut microbiome sample from the individual;
detecting Gammaproteobacteria, Granulicatella, and/or Alistipes levels in the second sample; and
comparing the levels of a given bacterium/bacteria in the second gut microbiome sample to levels of those same bacterium/bacteria in the first gut microbiome sample,
wherein if the Gammaproteobacteria and/or Granulicatella levels in the second sample are lower than the levels in the first sample and/or Alistipes levels in the second sample are higher than the levels in the first sample, continuing the dosage regimen for the individual.

93-97. (canceled)

98. The method according to claim 92 wherein the microbiome modulating compound is selected from the group consisting of: resistant potato starch, probiotic genera, species, and strains; prebiotics supporting growth of probiotic genera, species and strains; resistant starch from corn, tapioca, banana, grains, tubers and the like; fructooligosaccharides; galactooligosaccharides; xylooligosaccharides; mannanoligosaccharides; arabinoxylooligosaccharides; arabinogalactan polysaccharides; galactomannan polysaccharides; dietary changes that support the growth of probiotic bacteria; dietary treatments that increase the availability of resistant starch and/or prebiotics and/or other fermentation substrates to bacteria that inhibit the growth of Haemophilus, Pasteurellaceae, Enterobacteriaceae, Gammaproteobacteria, Granulicatella, Lachnospira, Oribacterium, Fusobacteriaceae not belonging to the genera Cetobacterium, Fusobacterium, Propionigenium, Psychrilyobacter, or u114 (hereafter ‘select Fusobacteriaceae’), Bacteroidaceae not belonging to the genera 5-7N15, Bacteroides, or BF311 (hereafter ‘select Bacteroidaceae’), Anaerostipes and/or Mogibacterium in the digestive tract; and antibiotics that target Haemophilus, Pasteurellaceae, Enterobacteriaceae, Gammaproteobacteria, Granulicatella, Lachnospira, Oribacterium, select Fusobacteriaceae, select Bacteroidaceae, Anaerostipes and/or Mogibacterium; mixed plant cell wall fibers; beta-glucans; resistant dextrins; resistant maltodextrins; limit dextrins; polydextrose; alginate; pectin polysaccharides; hydroxypropylmethylcellulose; chitin; chondroitin-containing compounds; and glucosamine-containing compounds.

99. (canceled)

100. The method according to claim 92 wherein the IBS-related parameter is selected from the group consisting of: abdominal pain, including bloating or abdominal distension, approximately one day per week, related to increases or decreases in pain with defecation, pain associated with changes in stool frequency, and/or pain associated with changes in stool shape; bloating with or without pain; diarrhea; constipation; belching; gas (ie. flatus); and/or reports of overall well-being, including mental wellness, anxiety, depression, fatigue, sleeplessness and the like.

101. The method according to claim 92 wherein the suitable period of time is from 1 week to 6 months.

102. (canceled)

103. The method according to claim 92 wherein the gut microbiome modulating compound is resistant potato starch.

104. The method according to claim 92 wherein the effective amount is 2 to 40 g per day of resistant potato starch.

105-164. (canceled)

165. A method for determining efficacy of a microbiome modulating of diarrhea in an individual at risk of developing or who has developed or who has IBS, said method comprising:
detecting Ruminococcus levels in a first gut microbiome sample from the individual at a first time point;
determining a first measurement of an IBS-related parameter of the individual at the first time point;
administering to the individual a microbiome modulating treatment on a dosage regimen for a suitable period of time;
following the suitable period of time, obtaining a second gut microbiome sample from the individual;
detecting Ruminococcus levels in the second sample;
determining a second measurement of the IBS-related parameter of the individual at the second time point;
comparing the levels of Ruminococcus in the second gut microbiome sample to levels of Ruminococcus in the first gut microbiome sample, and
comparing the first measurement of the IBS-related parameter and the second measurement of the IBS-related parameter,
wherein if the Ruminococcus levels in the second sample are higher than the Ruminococcus levels in the first sample, and the second IBS-related parameter is improved compared to the first IBS-related parameter, continuing the dosage regimen for the individual.

166-171. (canceled)

172. The method according to claim 165 wherein the microbiome modulating compound is selected from the group consisting of: resistant potato starch, probiotic genera, species, and strains; prebiotics supporting growth of probiotic genera, species and strains; resistant starch from corn, tapioca, banana, grains, tubers and the like; fructooligosaccharides; galactooligosaccharides; xylooligosaccharides; mannanoligosaccharides; arabinoxylooligosaccharides; arabinogalactan polysaccharides; galactomannan polysaccharides; dietary changes that support the growth of probiotic bacteria; dietary treatments that increase the availability of resistant starch and/or prebiotics and/or other fermentation substrates to Ruminococcus in the digestive tract; and antibiotics that target bacteria that inhibit the growth of Ruminococcus; mixed plant cell wall fibers; beta-glucans; resistant dextrins; resistant maltodextrins; limit dextrins; polydextrose; alginate; pectin polysaccharides; hydroxypropylmethylcellulose; chitin; chondroitin-containing compounds; and glucosamine-containing compounds.

173. The method according to claim 172 wherein the mixed plant cell wall fibers comprise two or more of the following plant cell wall fibers in varying proportions: cellulose, pectin, lignin, beta-glucan, and arabinoxylan regardless of source.

174. The method according to claim 166 wherein the IBS-related parameter is selected from the group consisting of: abdominal pain, including bloating or abdominal distension, approximately one day per week, related to increases or decreases in pain with defecation, pain associated with changes in stool frequency, and/or pain associated with changes in stool shape; bloating with or without pain; diarrhea; constipation; belching; gas (ie. flatus); and/or reports of overall well-being, including mental wellness, anxiety, depression, fatigue, sleeplessness and the like.

175. The method according to claim 165 wherein the suitable period of time is from 1 week to 6 months.

176. (canceled)

177. The method according to claim 165 wherein the gut microbiome modulating compound is resistant potato starch.

178. The method according to claim 165 wherein the effective amount is 2 to 40 g per day of resistant potato starch.

179-209. (canceled)
